(19) United States
(12) Patent Application Publication
(10) Pub. No.: US 2024/0189279 A1
(43) Pub. Date: Jun. 13, 2024

(54) IMMUNOMODULATORY COMPOSITIONS AND METHODS OF USE THEREOF

(71) Applicants: THE TEXAS A&M UNIVERSITY SYSTEM, College Station, TX (US); TRUSTEES OF TUFTS COLLEGE, Medford, MA (US)

(72) Inventors: Arul Jayaraman, College Station, TX (US); Kyongbum Lee, Medford, MA (US)

(21) Appl. No.: 18/536,002

(22) Filed: Dec. 11, 2023

Related U.S. Application Data
(60) Provisional application No. 63/386,906, filed on Dec. 11, 2022.
(60) Provisional application No. 63/513,155, filed on Jul. 12, 2023.

(51) Int. Cl. A61K 31/405; A61K 9/00; A61K 31/4045; A61P 1/16; A61P 11/00; A61P 31/14

(52) U.S. Cl. CPC A61K 31/405; A61K 9/0053; A61K 31/4045; A61P 1/16; A61P 11/00; A61P 31/14

(57) ABSTRACT

Provided here are immunomodulatory compositions for and methods of treatment of inflammation-associated diseases, such as liver disorders and acute respiratory distress syndrome (ARDS). The composition can be a tryptophan metabolite, such as indole 3-acetate, tryptamine, xanthurenic acid, or a derivative of any thereof. The methods can include administering the subject a therapeutically effective amount of the immunomodulatory composition provided herein. The liver disorders can be non-alcoholic fatty liver disease, steatosis, non-alcoholic steatohepatitis, and/or fibrosis. The ARDS can be associated with severe acute respiratory syndrome coronavirus 2 (SARS-CoV-2) infection.

### CROSS REFERENCE TO RELATED APPLICATIONS

This application claims the benefit of and priority to U.S. Provisional Application No. 63/386,906, filed Dec. 11, 2022, and U.S. Provisional Application No. 63/513,155, filed Jul. 12, 2023. The content of each of the foregoing applications is incorporated by reference herein in its entirety.

### TECHNICAL FIELD

The disclosure relates to certain compositions and methods of treatment of inflammation-associated diseases, such as inflammation-associated liver disorders and acute respiratory distress syndrome (ARDS).

### BACKGROUND

There is a need to develop safely and effectively treat inflammation-associated diseases. For example, non-alcoholic fatty liver disease (NAFLD) is the most common chronic liver disease in Western countries, with a prevalence rate of 21-25% in North America and Europe, and an even higher rate (20-30%) in parts of the Middle East. It is a multi-stage disease that can progress from liver steatosis (characterized by macrovesicular fat deposition), which is benign and reversible, to more severe forms of the disease such as non-alcoholic steatohepatitis (NASH) and fibrosis. Approximately 25% of individuals having liver steatosis develop NASH, which is characterized by liver inflammation, dysregulated lipid metabolism, and cell damage. A subset of NASH patients develops cirrhosis and even hepatocellular carcinoma. Factors leading to the progression from steatosis to NASH remain poorly understood, and there are currently no pharmacological treatments available for NASH. There thus is a need to develop new options to safely and effectively treat liver disorders, such as steatosis, NASH, and other inflammation-induced liver diseases.

For another example, ARDS is an inflammatory lung injury caused by buildup of fluids in alveoli in the lungs. This prevents the lungs from filling up with air and causes dangerously low oxygen levels in the blood (hypoxemia). Patients develop ARDS as a result of another disease, such as sepsis, pneumonia, the coronavirus (COVID-19) and other conditions, or a major injury. ARDS is a major factor contributing to the mortality of patients infected with SARS-CoV-2. There is a significant correlation between ARDS and inflammatory cytokines in severely ill COVID-19 patients. In these patients, inflammatory cytokines remain elevated even after the virus clears from the lungs. The cytokines implicated in this “cytokine storm” include: IL-1, IL-2, IL-4, IL-6, IL-7, IL-10, IL-12, IL-13, IL-17, M-CSF, G-CSF, GM-CSF, IP-10, IFN-γ, MCP-1, MIP 1-α, hepatocyte growth factor (HGF), TNF-α, and vascular endothelial growth factor (VEGF). Interestingly, this list encompasses both pro- and anti-inflammatory interleukins that are conventionally associated with stimulating and resolving inflammatory responses, respectively. The extent to which the aforementioned cytokines increase in a patient as a result of SARS-Cov-2 infection depends on a number of factors, including the types of interventions received by the patient. However, a common denominator in many severely ill patients is that there is a significant correlation between a sustained elevation in IL-6 post-infection and poor outcome (higher mortality or longer recovery period). This trend is consistent with improved patient outcomes resulting from antibody therapy targeting IL-6 signaling. A notable example is the use of Tocilizumab, a humanized antibody against the IL-6 receptor. A recent study by Oxford University has found a reduction in the mortality rate of sick patients on respiratory support when they are treated with dexamethasone, a corticosteroid drug with anti-inflammatory and immunosuppressive effects. On the other hand, the use of anti-inflammatory and immunosuppressive drugs poses significant risks, and the data to support their use and comprehensively assess their adverse effects during use in this disease, is unclear at this time. Blocking the inflammatory cytokine cascade early on in disease could compromise the body's ability to fight the inciting infection. Even if given later in the disease course, it could potentially place the patient at risk of secondary infection. There thus is a need to develop new options to safely and effectively treat the cytokine storm in COVID-19 patients.

### SUMMARY

Provided here are compositions and methods of treating inflammation of the liver, the intestine, or the lung. In an embodiment, administering to the human subject the therapeutically effective amount of the composition according to the methods provided herein decreases inflammation.

Provided here are compositions and methods of treating inflammation-associated diseases. In an embodiment, the method includes administering to a human subject a therapeutically effective amount of a composition containing tryptophan metabolites or derivatives thereof. In an embodiment, the method includes administering to a human subject a therapeutically effective amount of a composition containing indole 3-acetate or a derivative thereof. In an embodiment, the method includes administering to a human subject a therapeutically effective amount of a composition containing tryptamine or a derivative thereof. In an embodiment, the method includes administering to a human subject a therapeutically effective amount of a composition containing xanthurenic acid or a derivative thereof. In an embodiment, the composition is administered to the human subject orally. In an embodiment, the composition is formulated as liquid, powder, tablet, paste, capsule, or gel.

In an embodiment, the inflammation-associated disease to be treated is a liver disorder. The liver disorder can be NAFLD at any one of the stages, including but not limited to steatosis, NASH, and fibrosis. In an embodiment, administering to the human subject the therapeutically effective amount of the composition according to the methods provided herein increases liver total bile acids, decreases primary bile acids; and/or decreases free fatty acid concentration in liver bile acid. In an embodiment, the inflammation-associated disease to be treated is acute respiratory distress syndrome (ARDS). The ARDS can result from a viral infection. In an embodiment, the viral infection is caused by severe acute respiratory syndrome coronavirus 2 (SARS-CoV-2).

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. To facilitate this description, like reference numerals designate like structural elements or procedures in a method. Embodiments are illustrated by way of example and not by way of limitation in the figures of the accompanying drawings.

FIGS. 1A-1F are graphical and photographical representations demonstrating that oral administration of I3A alleviates diet induce hepatic steatosis and inflammation. FIG. 1A and FIG. 1B are graphical representations of fecal (at weeks 8 and 16) and liver (at week 16) concentrations, respectively, of I3A in male B6 129SF1/J mice in control low-fat diet (CN), Western diet (WD), Western diet with low-dose I3A (WD-50), and Western diet with high-dose I3A (WD-100). FIG. 1C is a graphical representation of the serum alanine aminotransferase (ALT) levels in mice at weeks 10 and 16. FIG. 1C is a graphical representation of the liver triglyceride (TG) levels at week 16. Data shown are TG concentrations (mg/dl) normalized to corresponding tissue DNA contents (μg DNA). FIG. 1E is a set of photographical representations of representative liver sections stained with hematoxylin-eosin (H&E). FIG. 1F is a graphical representation of the histology score for steatosis (upper left panel), hepatocyte ballooning (upper right panel), and lobular inflammation (lower left panel) from 3 stained sections per animal. H&E-stained liver sections were evaluated by an expert pathologist using the NASH CRN and fatty liver inhibition of progression (FLIP) consortia criteria. Data shown are mean±SEM (n=10 per group). *: p<0.05, **: p<0.01, ***: p<0.001 using Wilcoxon rank sum test.

FIG. 2A is a set of graphical representations of the effect of I3A on inflammatory cytokine levels in livers of male B6/129 mice (n=10 mice per group) at week 16. Animals were fed either a control (CN) or Western diet (WD). A randomly selected subset of WD mice were given an average daily dose of 50 (WD-50) or 100 (WD-100) mg of I3A per kg body wt. Cytokines were measured using a bead-based multiplex assay in homogenized, lyophilized, and defatted liver samples. Dots and bars show individual liver values and means.

FIG. 2B is a set of graphical representations of liver total bile acid concentration (left panel), abundance of primary bile acids relative to total (middle panel), and abundance of conjugated primary bile acids relative to primary bile acids (right panel) with I3A supplementation. FIG. 2C is a set of graphical representations of the primary, secondary, and conjugated primary bile acid levels in the liver with I3A supplementation. CN: control mice, WD-50: WD mice with 50 mg/kg body weight through drinking water, WD-100: WD mice with 100 mg/kg body weight through drinking water. Data shown are mean±SEM. *: p<0.05, **: p<0.01, ***: p<0.001 using Wilcoxon rank sum test.

FIGS. 3A and 3B demonstrate that I3A administration partially reverses diet-induced metabolome alterations in the liver. FIG. 3A is a set of scatter plots of latent variable projections from PLS-DA of untargeted metabolomics data features. Comparison of all four experimental groups (left panel), CN vs. WD group (middle panel), and WD vs. WD-50 and WD-100 groups (right panel). FIG. 3B is a set of principal coordinate analysis of liver metabolome with I3A supplementation. FIG. 3C is a heatmap of significant metabolite features (p<0.05) based on statistical comparisons of treatment groups (CN vs. WD, WD vs. WD-50, and WD vs WD-100) using non-parametric ANOVA and Wilcoxon-rank sum test. FIG. 3D is a heat map showing hierarchical clustering of the different groups. FIG. 3E is a correlation between TG and I3A levels in livers from the WD-100 group. CN: control mice, WD-50: WD mice with 50 mg/kg body weight through drinking water, WD-100: WD mice with 100 mg/kg body weight through drinking water.

FIGS. 4A-4C demonstrate that I3A administration partially reverses diet-induced proteome alterations in the liver. FIG. 4A is a set of scatter plots of latent variable projections from PLS-DA of confidently identified proteins. Comparison of all four experimental groups (upper left panel), CN vs WD group (upper right panel), and WD vs. WD-50 and WD-100 groups (lower left panel). FIG. 4B is a heatmap of significant proteins having Variable Importance in Projection score >1.2. The proteins were clustered using k-means. FIG. 4C is a tabular presentation of the pathway enrichment analysis of significant proteins differentially abundant in CN vs. WD comparison (upper panel) and WD-100 vs. WD comparison (lower panel). GeneRatio divides the number of significantly altered proteins that are in the pathway by the total number of significantly altered proteins. BGRatio divides the number of proteins that are in the pathway by the number of all detected proteins. The p-value was calculated using Fisher's exact test.

FIGS. 5A-5C demonstrate that I3A administration reduces the levels of enzymes in fatty acid transport, de novo lipogenesis and β-oxidation. FIG. 5A is a set of graphical representations of the abundance of fatty acid translocase (CD36) and fatty acid synthase. FIGS. 5B and 5C is a set of graphical representations of the mitochondrial and peroxisomal fatty acid oxidation enzymes. Data shown are mean±SEM. *: p<0.05, **: p<0.01 using Wilcoxon rank sum test

FIGS. 6A-6B demonstrate that I3A administration reverses WD-induced reduction in liver p-AMPK and AMPK. FIG. 6A is a photograph of a Western blot analysis of the levels of p-AMPK and AMPK in liver tissue at week 16. FIG. 6B is a set of graphical representations of the ratios of p-AMPK (left panel) and AMPK (right panel) to β-actin. The ratios were determined based on the p-AMPK and AMPK band intensities quantified using Image Lab (Bio-Rad) and normalized to the loading control (β-actin). Data shown are mean±SEM. *: p<0.05, **: p<0.01 using Wilcoxon rank sum test.

FIGS. 7A-7D demonstrate that I3A suppresses macrophage inflammation in vitro in an AMPK dependent manner. FIG. 7A is a photograph of a Western blot analysis of the expression levels of p-AMPK and total AMPK in RAW 264.7 macrophages pre-treated with either I3A or vehicle (DMF) control followed by stimulation with palmitate and LPS. FIG. 7B is a set of graphical representations of the fold-changes in p-AMPK and total AMPK. Fold-changes were calculated relative to the DMF and no palmitate and LPS stimulation condition. The band intensities were quantified and normalized to loading control (β-actin) by using Image Lab (Bio-Rad). FIG. 7C is a set of graphical representations of the expression levels of TNFα and IL-1β in RAW 264.7 cells treated with p-AMPK activator AICAR, followed by stimulation with palmitate and LPS. FIG. 7D is a set of graphical representations of the expression levels of TNFα (top row) and IL-1β(bottom row) in RAW 264.7 cells transduced with non-targeted control siRNA (left panels) or Prkaa1 siRNA (right panels), pre-treated with I3A, and then stimulated with palmitate and LPS. Data show are mean±SEM from three independent cultures with three biological replicates. *: p<0.05, **: p<0.01, ***: p<0.001 using Student's t-test.

FIGS. 8A-8B are proposed models for the effects of I3A on hepatic lipid metabolism and inflammation. When mice are fed with a WD (FIG. 8A), TG and FFAs accumulate in the liver due to increased uptake of fatty acids. This also leads to increased β-oxidation in the mitochondria and peroxisomes. In liver macrophages, the increase in FFAs, possibly in conjunction with circulating endotoxins (e.g., LPS) stimulate production of inflammatory cytokines. When mice fed the WD are treated with I3A (FIG. 8B), both TG and FFAs decrease in the liver. Rather than impact fatty acid uptake, I3A treatment reduces de novo lipogenesis through a downregulation of Fasn, while also reducing both mitochondrial and peroxisomal β-oxidation. In macrophages, I3A attenuates fatty acid and LPS stimulated production of inflammatory cytokines through activation of AMPK.

FIG. 9A presents the study design. Three groups of male B6 129SF1/J mice (n=10 for each group) were fed ad libitum a Western diet (WD) and a sugar water (SW) solution while a fourth group was given normal chow diet. After 8 weeks, the three groups of WD-fed mice were randomly selected for treatment with vehicle (WD group) or low (WD-50 group) or high dose (WD-100 group) of I3A for an additional 8 weeks. The fourth group (CN) was continued on low-fat diet calorie matched with the WD. FIG. 9B presents the sampling scheme. FIG. 9C is a graphical representation of the quantification of I3A in serum. FIG. 9D is a graphical representation of the body weight increase of the four groups, normalized to week 8 body weight when I3A administration was started. Data shown are mean±SEM. *: p<0.05, **: p<0.01, WD-100 group compared to WD group using Wilcoxon rank sum test. #: p<0.05, WD-50 group compared to WD group using Wilcoxon rank sum test.

FIGS. 10A-10B demonstrate that I3A reduces liver inflammatory cytokine expression and total FFA concentration. FIG. 10A is a set of graphical representations of the inflammatory cytokines in liver tissue at week 16. FIG. 10B is a set of graphical representations of the FFAs in diet, serum and liver samples. Data shown are mean±SEM. *: p<0.05, **: p<0.01, ***: p<0.001 using Wilcoxon rank sum test.

FIGS. 11A-11E demonstrate that I3A administration does not significantly alter the fecal microbial community. FIG. 11A is a set of graphical representations of the alpha diversity of the fecal microbiome from CN, WD, WD-50, and WD-100 groups. FIG. 11B is a set of Analysis of Similarities (ANOSIM) comparison for CN vs. WD group and WD vs. WD-50 vs. WD-100 groups. FIGS. 11C and 11D are graphical representations of the phylum and genus level relative abundance of the fecal microbial community members. *: p<0.05, ***: p<0.001 using Wilcoxon rank sum test. FIG. 11E is a set of graphical representations of the LDA score for CN vs. WD group and WD vs. WD-100 groups

FIGS. 12A-12B demonstrate that I3A administration does not significantly alter the fecal metabolome. FIG. 12A is a set of score plots showing the first two principal components for all four experimental groups (upper left panel), CN vs. WD group (upper right panel), and WD vs. WD-50 and WD-100 groups (lower left panel). Numbers in the parentheses of axis titles show percent of variance explained by the corresponding principal component. Ellipses circumscribe 95% confidence regions for the experimental groups assuming Gaussian distribution of component scores. FIG. 12B is a heatmap of fecal microbiome metabolite features detected in all treatment groups. Rows and columns are features and treatment groups, respectively. The features were clustered using k-means.

FIG. 13 shows the Principal Component Analysis of liver proteome after I3A administration. FIG. 13 is a set of score plots showing the first two principal components for all four experimental groups (upper left panel), CN vs. WD group (upper right panel), and WD vs. WD-50 and WD-100 groups (lower left panel). Numbers in the parentheses of axis titles show percent of variance explained by the corresponding principal component. Ellipses represent 95% confidence regions for the experimental groups assuming Gaussian distribution of component scores.

FIGS. 14A-14B demonstrate that I3A administration reduces the levels of antioxidant enzymes—Catalase (FIG. 14A) and glutathione peroxidase-1 (FIG. 14B) for the four experimental groups. Data shown are mean±SEM. **: p<0.01 using Wilcoxon rank sum test.

FIGS. 15A-15C demonstrate that Prkaa1 siRNA reduces AMPK expression in macrophages. FIG. 15A is a graphical representation of the levels of prkaa1 mRNA in RAW 264.7 cells transfected with prkaa1 or non-targeted control siRNA for 24 h, followed by incubation for an additional 24, 48, and 72 h. The expression level of prkaa1 is normalized to that of the housekeeping gene β-actin. FIG. 15B is a photograph of a Western blot analysis of p-AMPK and total AMPK from cells treated with the different siRNA. A representative blot is shown. FIG. 15C is a set of graphical representations of the quantified intensities of p-AMPK and total AMPK bands normalized to loading control (β-actin).

FIGS. 16A-16C demonstrate that I3A's anti-inflammatory effects in macrophages are independent of AhR activation. FIG. 16A is a photograph of a Western blot analysis of the expression level of AhR in RAW264.6 and AML12 cells. Raw 264.7 cells were treated with 1 mM I3A (or DMF solvent control) for 4 h, then stimulated with 300 μM Palmitate for 18 h and 10 ng/ml LPS for 6 h (two-hits model). The AhR inhibitor CH223191 (5 μM) or DMSO control were added 10 min before I3A treatment. Total RNA were isolated from the cells and FIG. 16B is a set of graphical representations of the expression of TNFα and IL-1β as measured with qRT-PCR. FIG. 16C is a set of graphical representations of the TNFα and IL-1β expression when plotted as fold change normalized to the DMF control group. Data presented as the mean±SEM. ***: p<0.001 using Student's t-test.

FIG. 17 is diagram of the untargeted proteomic data analysis workflow.

FIG. 18 is a set of photographic images of differentiated HIE expressing small intestinal epithelial markers sucrose isomaltase (FIG. 18A), chromagranin A (ChgA) (FIG. 18B), lysozyme (FIG. 18C), and mucin 2 (Muc2) (FIG. 18D).

### DETAILED DESCRIPTION

The present disclosure describes various embodiments related to certain immunomodulatory compounds and use of these compounds for treatment of liver disorders. In the following description, numerous details are set forth in order to provide a thorough understanding of the various embodiments. Before the present methods and compositions are described, it is to be understood that these embodiments are not limited to particular methods or compositions described, as such may, of course, vary. It is also to be understood that the terminology used herein is for the purpose of describing particular embodiments only, and is not intended to be limiting, as the scope of the present embodiments will be limited only by the appended claims. The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

As used herein, a “therapeutically effective amount” is an amount of an active ingredient (e.g., immunomodulatory compounds described here or derivatives thereof) or a pharmaceutically acceptable salt thereof that eliminates, ameliorates, alleviates, or provides relief of the symptoms for which it is administered. As used herein, the terms “treatment,” “treating,” and “treat” refer to any indicia of success in the amelioration of an injury, disease, or condition, including any objective or subjective parameter such as abatement; remission; diminishing of symptoms or making the injury, disease, or condition more tolerable to the subject; slowing in the rate of degeneration, decline, or other disease progression; making the final point of degeneration less debilitating; and/or improving a subject's physical or mental well-being. The terms “treatment,” “treating,” and “treat” also refer to preventing, stopping, or delaying onset of an injury, disease, or condition, including any objective or subjective parameter such as symptoms and signs.

The terms “administer,” “administering,” and “administration” refer to introducing a compound, a composition, or an agent (e.g., immunomodulatory compounds described here or derivatives thereof) into a subject or subject, such as a human. As used herein, the terms encompass both direct administration, e.g., self-administration or administration to a subject by a medical professional, and indirect administration, such as the act of prescribing a compound, composition, or agent. As used herein, the term “subject” refers to an individual (e.g., a human or a non-human mammal) having or prone to a condition that can be treated by administration of an immunomodulatory compound as provided herein. In some embodiments, subject has an inflammation-associated disease or condition. For example, in some embodiments, a subject has an acute respiratory distress syndrome. In some embodiments, a subject has a liver disorder, such as NAFLD, steatosis, steatohepatitis, and other inflammation-associated liver diseases.

Embodiment disclosed here are compositions and methods of treating inflammation-associated diseases and conditions. Compositions provided herein contain immunomodulatory compounds, such as a tryptophan metabolite or a derivative thereof, such as indole 3-acetate, tryptamine, xanthurenic acid, or a derivative of any thereof, which can be safely administered to modulate the hyperinflammatory response in an inflammation-associated disease such as steatosis, steatohepatitis, and ARDS.

### A. Compositions and Methods for Treating Liver Disorder

Provided here are compositions and methods of treating inflammation-associated liver disorders. The composition can be an immunomodulatory compound, such as tryptophan (Trp) derived microbial metabolites that attenuate the production of cytokines implicated in the inflammation-associated liver disease. The tryptophan metabolite can include indole 3-acetate (I3A), a Trp metabolite that modulates the production of several inflammatory cytokines, or a derivative thereof. The tryptophan metabolite can include tryptamine (TA) or a derivative thereof. The tryptophan metabolite can also include xanthurenic acid (XA) or a derivative thereof. These immunomodulatory compounds can attenuate inflammation associated with the liver disease.

In an embodiment, the method includes administering to a human subject a therapeutically effective amount of a composition containing tryptophan metabolites or derivatives thereof. In an embodiment, the method includes administering to a human subject a therapeutically effective amount of a composition containing indole 3-acetate or a derivative thereof. In an embodiment, the method includes administering to a human subject a therapeutically effective amount of a composition containing tryptamine or a derivative thereof. In an embodiment, the method includes administering to a human subject a therapeutically effective amount of a composition containing xanthurenic acid or a derivative thereof. The liver disorders can be NAFLD at any one of the stages, including but not limited to steatosis, NASH, and fibrosis.

NAFLD is the most common chronic liver disease in Western countries. There is growing evidence that dysbiosis of the intestinal microbiota and disruption of microbiota-host interactions contribute to the pathology of NAFLD. Gut microbiota derived tryptophan metabolite indole-3-acetate (I3A) was decreased in both cecum and liver of high-fat diet-fed mice and attenuated the expression of inflammatory cytokines in macrophages and TNF-a and fatty acid induced inflammatory responses in an aryl-hydrocarbon receptor (AhR) dependent manner in hepatocytes. Western diet (WD)-fed animals given sugar water (SW) with I3A showed dramatically decreased serum ALT, hepatic TG, liver steatosis, hepatocyte ballooning, lobular inflammation, and hepatic production of inflammatory cytokines, compared to WD-fed animals given only SW. Metagenomic analysis show that I3A administration did not significantly modify the intestinal microbiome, suggesting that I3A's beneficial effects likely reflect the metabolite's direct actions on the liver. Administration of I3A partially reversed WD induced alterations of liver metabolome and proteome, notably, decreasing expression of several enzymes in hepatic lipogenesis and β-oxidation. AMP-activated protein kinase (AMPK) mediates the anti-inflammatory effects of I3A in macrophages. The potency of I3A in alleviating liver steatosis and inflammation clearly demonstrates its therapeutic modality for preventing the progression of steatosis to NASH.

There is growing evidence that dysbiosis of the intestinal microbiota and disruption of microbiota-host interactions contribute to the pathology of NASH. Certain shifts in the intestinal microbiota community composition, e.g., expansion of the phyla Verrucomicrobia and Proteobacteria, correlate with NASH in both human and animal studies. One potential mechanism linking intestinal microbiota dysbiosis and NASH is compromised intestinal barrier integrity, which promotes the translocation of bacterial products from the lumen to circulation. This can contribute directly and indirectly (through intestinal inflammation) to liver inflammation. Additionally, microbial dysbiosis alters the balance of bioactive metabolites produced by gut bacteria such as bile acids, short chain fatty acids and aromatic amino acid derivatives, which have been shown to impact liver metabolism and inflammation in NAFLD through host receptor mediated pathways.

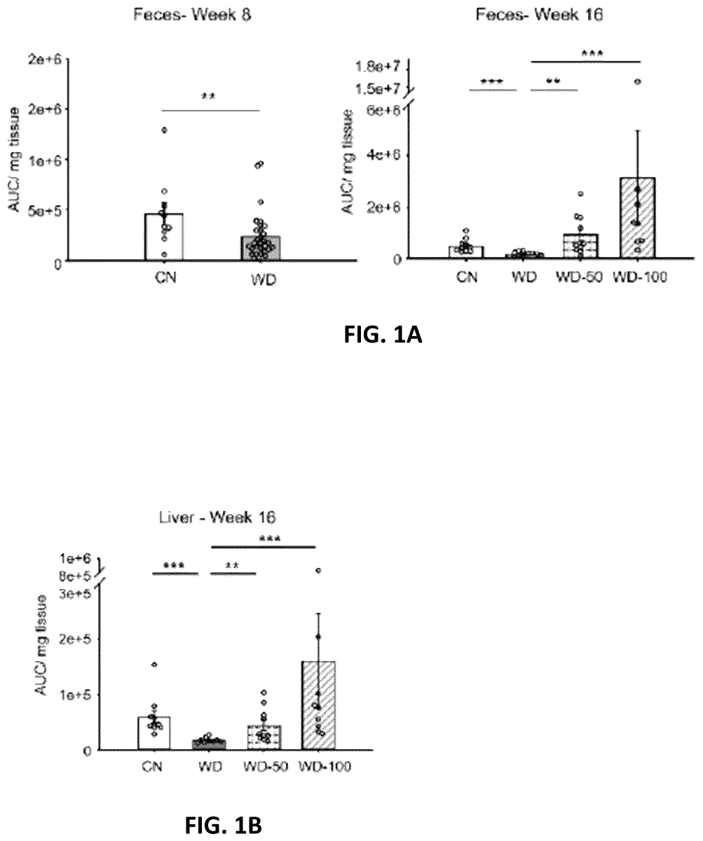

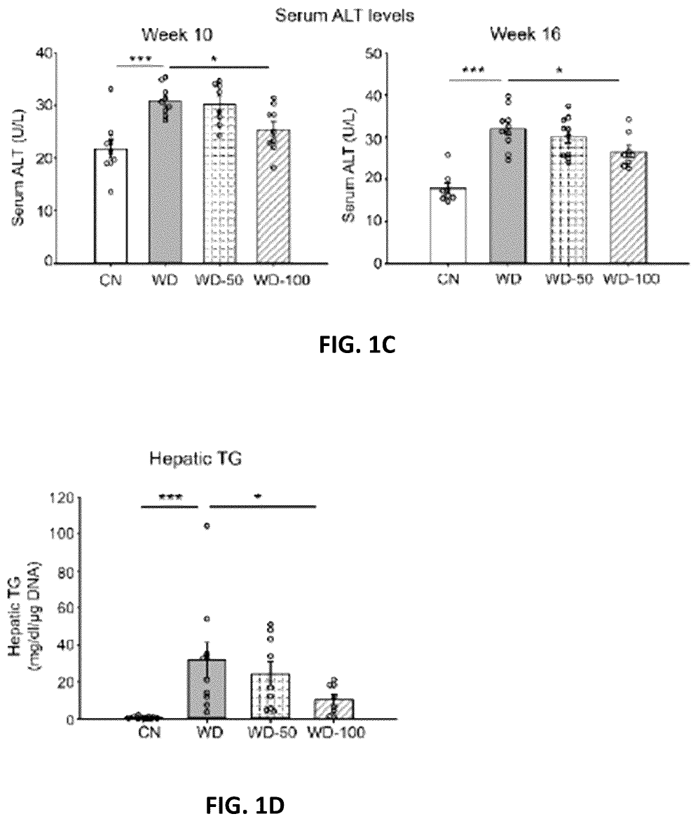

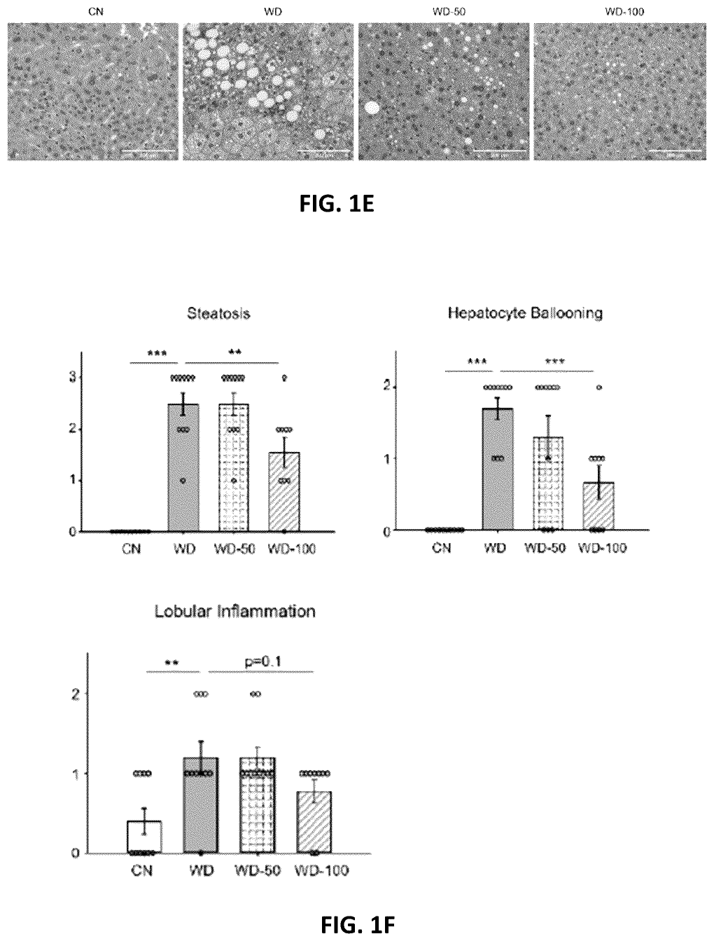

I3A modulates inflammatory cytokine expression in macrophages and lipid metabolism in hepatocytes. The effects of I3A to mitigate liver steatosis and other NAFLD features in vivo were investigated. FIGS. 1A-1F are graphical and photographical representations demonstrating that oral administration of I3A alleviates diet induce hepatic steatosis and inflammation. FIG. 1A and FIG. 1B are graphical representations of fecal (at weeks 8 and 16) and liver (at week 16) concentrations, respectively, of I3A in male B6 129SF1/J mice in control low-fat diet (CN), Western diet (WD), Western diet with low-dose I3A (WD-50), and Western diet with high-dose I3A (WD-100). Results show that oral administration of I3A protects against liver injury in a dose-dependent manner (FIG. 1C), attenuates liver TG accumulation (FIG. 1D), and reduces steatosis, hepatocyte ballooning, and lobular inflammation (FIGS. 1E and 1F), suggesting that administration of I3A altered both metabolic and inflammation pathways in the liver. FIG. 1C is a graphical representation of the serum alanine aminotransferase (ALT) levels in mice at weeks 10 and 16. FIG. 1C is a graphical representation of the liver triglyceride (TG) levels at week 16. Data shown are TG concentrations (mg/dl) normalized to corresponding tissue DNA contents (μg DNA). FIG. 1E is a set of photographical representations of representative liver sections stained with hematoxylin-eosin (H&E). FIG. 1F is a graphical representation of the histology score for steatosis (upper left panel), hepatocyte ballooning (upper right panel), and lobular inflammation (lower left panel). H&E-stained liver sections were evaluated by an expert pathologist using the NASH CRN and fatty liver inhibition of progression (FLIP) consortia criteria. Data shown are mean±SEM (n=10 per group). *: p<0.05, **: p<0.01, ***: p<0.001 using Wilcoxon rank sum test.

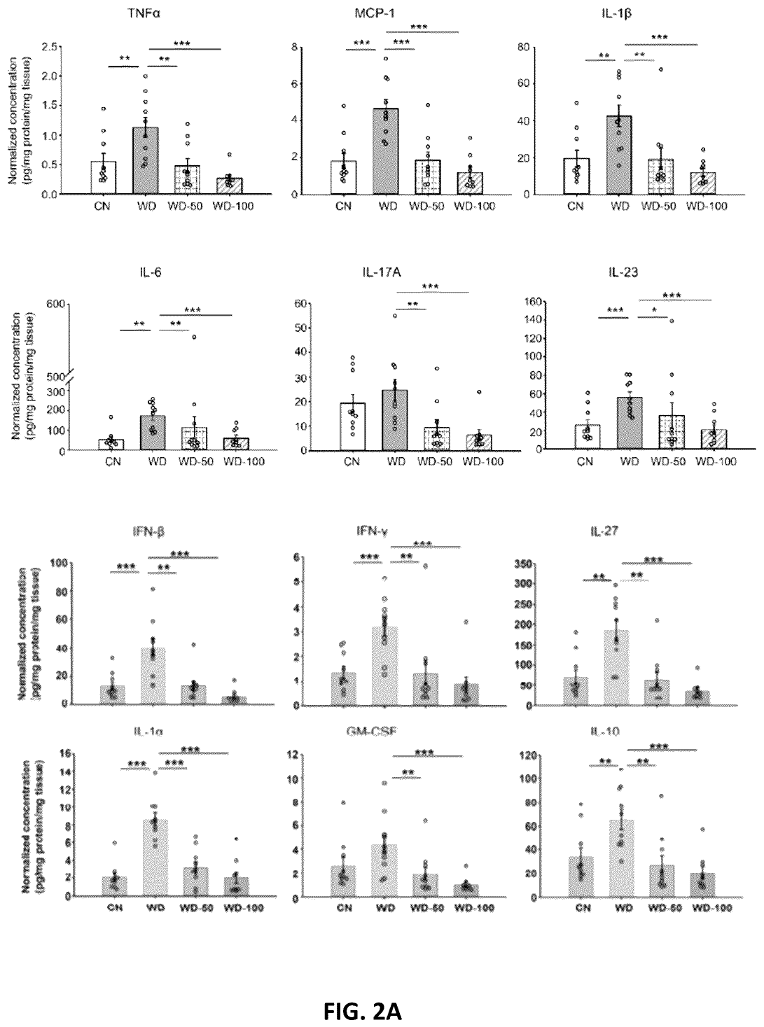

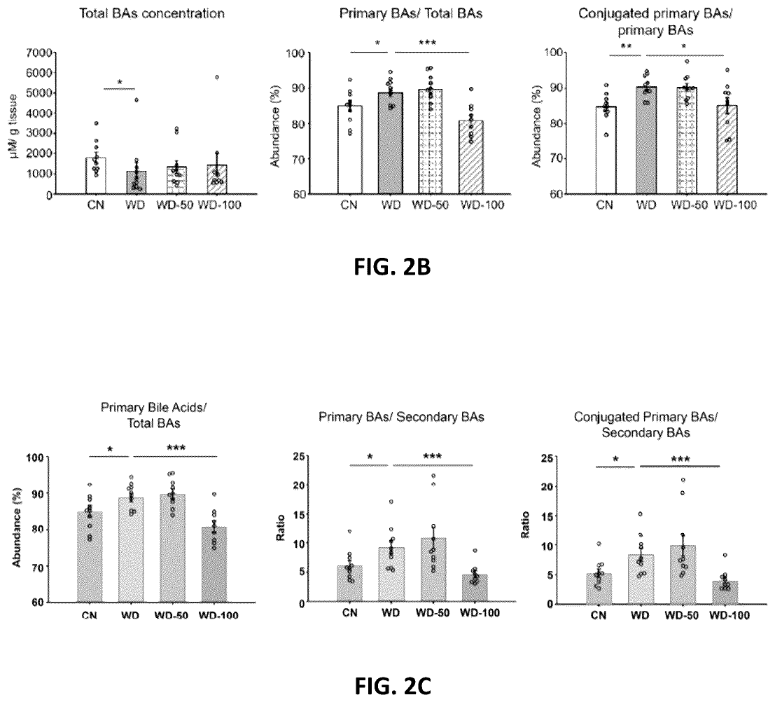

Production of pro-inflammatory cytokines such as TNFα, IL-1β, MCP-1 and IL-6 by resident macrophages (Kupffer cells) signals recruitment and infiltration of monocytes into the liver. These cytokines also promote the differentiation of monocytes into pro-inflammatory macrophages, which in turn exacerbates dysregulation of hepatic lipid metabolism. Consistent with the lobular inflammation score, there was a significant increase in the expression of inflammatory cytokines in livers of the WD group (FIG. 2A). FIG. 2A is a set of graphical representations of inflammatory cytokines in liver tissue at week 16, demonstrating that I3A administration reverses WD induced alterations in liver inflammatory cytokines and bile acids. Remarkably, treatment of WD fed mice with I3A dose-dependently reduced the expression of every cytokine in the 13-member panel, including IL-10. The downregulation of IL-10 in I3A treated mice could be a result of reduced hepatic inflammation (i.e., feedback regulation of IL-10 production).

Alteration of bile acid metabolism could be a biomarker for NAFLD. Animals in the WD group had a lower total concentration of liver BAs and higher proportion of primary BAs compared to CN (FIG. 2B). FIG. 2B is a set of graphical representations of liver total bile acid concentration (left panel), abundance of primary bile acids relative to total (middle panel), and abundance of conjugated primary bile acids relative to primary bile acids (right panel). FIG. 2C is a set of graphical representations of the primary, secondary, and conjugated primary bile acid levels in the liver with I3A supplementation. The trend is consistent with previous studies comparing BA profiles between NASH patients and healthy controls. I3A treatment reversed this trend in a dose-dependent manner.

The observation that the fecal microbial community composition was not significantly altered by I3A administration suggests that the hepatoprotective effects of I3A are likely due to the metabolite's direct action on the host, rather than the gut microbiota composition. One caveat to this interpretation is the shallow resolution of 16S rRNA sequencing. It is possible that I3A administration altered the microbiota composition at the species level. It is also possible that I3A could affect the metabolism of the microbiota without altering its composition. A recent study showed that a diet containing high levels of methionine and cysteine protected mice against chronic kidney disease by reducing uremic toxins through post-translational modification of gut bacterial enzymes. To test the possibility that I3A administration altered the intestinal metabolome, untargeted metabolomics experiments were performed on fecal samples. The results showed no significant impact of I3A on the fecal metabolomes of WD mice. While further studies are warranted to fully elucidate the impact of I3A administration on the microbiome and microbial gene expression in the intestine, these results suggest that the partial reversal of WD-induced changes in liver metabolome and proteome by I3A treatment is due to the metabolite's direct action on the liver.

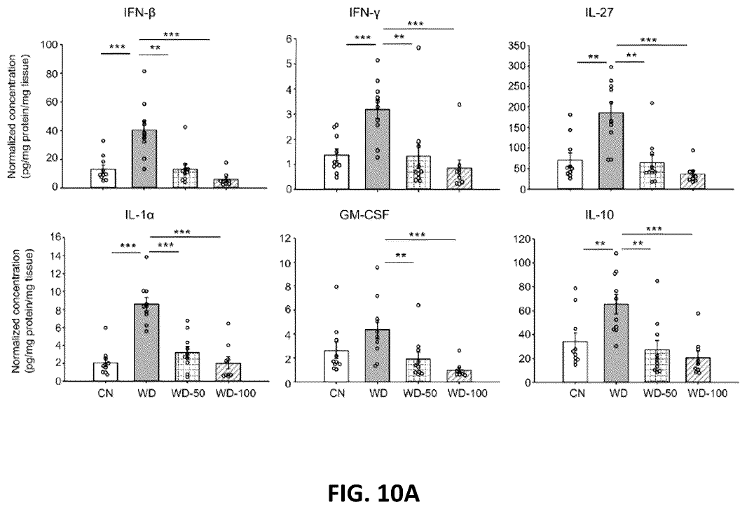

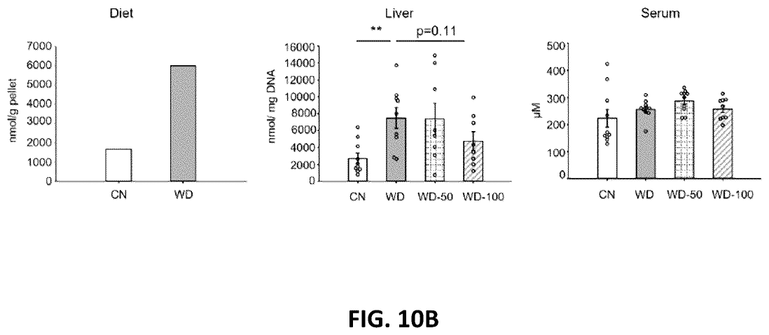

Analysis of enzymes in lipid transport, synthesis, and mobilization showed that WD and I3A impacted different facets of lipid metabolism. Targeted proteomics detected a 6-fold increase in fatty acid translocase CD36 in the WD group compared to CN, suggesting that the elevation of liver FFAs in the WD group (FIG. 10B) could be due to increased uptake. I3A did not affect CD36 abundance, but significantly decreased Fasn by 86%, the rate limiting enzyme in de novo lipogenesis. The significant decrease in liver TG in the WD-100 group could reflect a downregulation of de novo lipid synthesis in the liver.

Liver fatty acid oxidation (FAO) enzymes also showed different responses to WD and I3A. Acetyl-CoA carboxylase 2 (Acab) is a mitochondrial regulatory enzyme that produces malonyl-CoA, which inhibits carnitine palmitoyltransferase 1 (CPT1). Downregulation of Acab in the WD group compared to CN suggests increased CPT1 activity and hence elevated long-chain FA (LCFA) transport into the mitochondria. Other mitochondrial fatty acid oxidation (FAO) enzymes also showed a trend towards upregulation in the WD group compared to CN. Upregulation of FAO by WD was significant in the peroxisomes, the initial site of very long-chain FA oxidation. The literature is conflicted on FAO in human subjects with NAFLD or NASH. Depending on the study, enhanced, unchanged, or decreased FAO has been reported. The different results may reflect varying severity of the disease (degree of steatosis or steatosis vs. NASH) and variations in FAO capacity across individual subjects. One study showed that the expression of genes in peroxisomal and mitochondria β-oxidation was higher in patients with more severe steatosis compared to patients with less sever steatosis or healthy control. Another study found that plasma β-hydroxybutyrate, an indirect measure of FAO, was higher in patients with NASH compared to simple steatosis or healthy control. Interestingly, administration of I3A significantly reduced the abundance of both mitochondrial and peroxisomal FAO enzymes. Increased FAO and concomitant ROS generation can overwhelm cellular antioxidant defenses, inducing oxidative stress. Although there is a lack of consensus regarding FAO, studies consistently report elevated markers of oxidative stress in human subjects with steatosis. In this regard, I3A may protect from oxidative stress by reducing FAO. Oxidative stress induced enzymes CAT and GPx were upregulated in the WD group compared to CN and downregulated in the WD-100 group compared to the WD group. It should be noted that all the biochemical parameters related to lipid metabolism were assessed at termination of the mouse experiments. It is possible that the downregulation of FAO enzymes in I3A treated mice reflects a response to reduced lipid accumulation. Further longitudinal studies are warranted to determine the (likely) dynamic effects of I3A on the liver lipid metabolism.

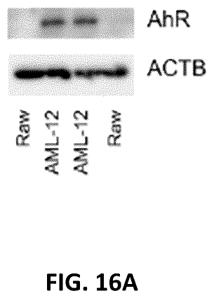

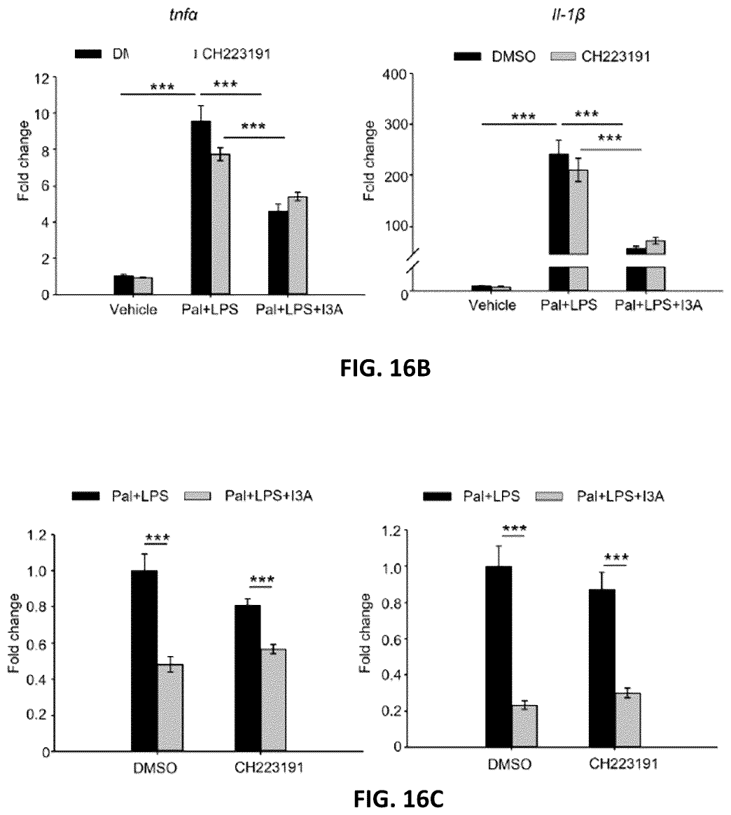

Recent studies have demonstrated that tryptophan derived gut bacterial metabolites, including I3A, are AhR agonists in intestinal epithelial cells, hepatocytes, and immune cells. I3A decreased the expression of Fasn and its transcriptional regulator SREBP-1c in hepatocytes in an AhR dependent manner. In macrophages, however, I3A's effects on macrophages were independent of AhR activity, as addition of the AhR inhibitor did not alter I3A's effects. FIGS. 16A-16C demonstrate that I3A's anti-inflammatory effects in macrophages are independent of AhR activation. FIG. 16A is a photograph of a Western blot analysis of the expression level of AhR in RAW264.6 and AML12 cells. Raw 264.7 cells were treated with 1 mM I3A (or DMF solvent control) for 4 h, then stimulated with 300 μM Palmitate for 18 h and 10 ng/ml LPS for 6 h (two-hits model). The AhR inhibitor CH223191 (5 μM) or DMSO control were added 10 min before I3A treatment. Total RNA were isolated from the cells and FIG. 16B is a set of graphical representations of the expression of TNFα and IL-1β as measured with qRT-PCR. FIG. 16C is a set of graphical representations of the TNFα and IL-1β expression when plotted as fold change normalized to the DMF control group. Data presented as the mean±SEM. ***: p<0.001 using Student's t-test.

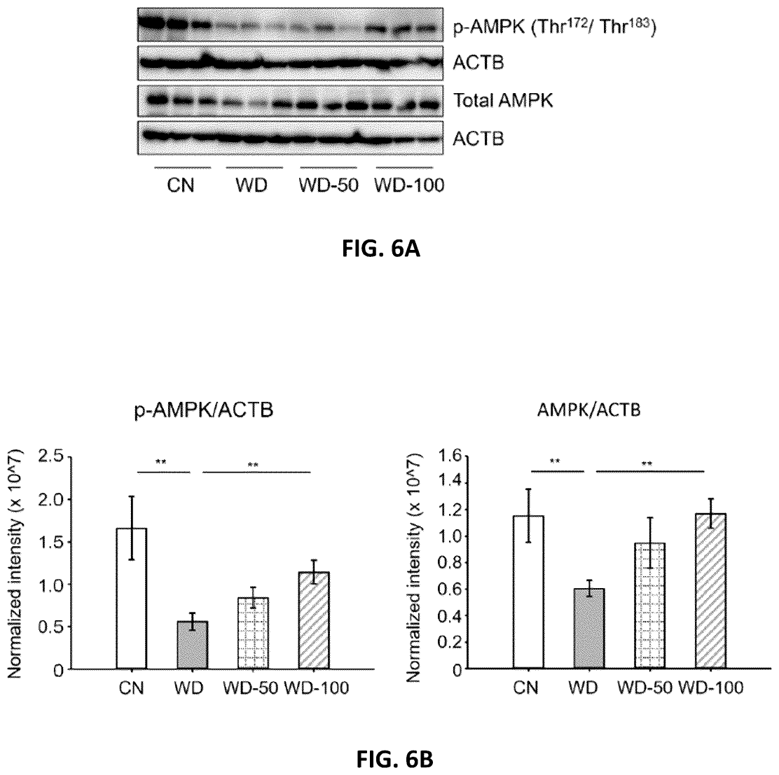

Based on studies in HFD-fed mice showing dysregulation of liver AMPK activity and its association with increased lipid accumulation, the role of AMPK in mediating the response to I3A was investigated. Both liver AMPK expression and phosphorylation were reduced in WD-fed mice relative to CN, and that administration of I3A rescued both AMPK expression and phosphorylation in a dose dependent manner. FIGS. 6A-6B demonstrate that I3A administration reverses WD-induced reduction in liver p-AMPK and AMPK. FIG. 6A is a photograph of a Western blot analysis of the levels of p-AMPK and AMPK in liver tissue at week 16. FIG. 6B is a set of graphical representations of the ratios of p-AMPK (left panel) and AMPK (right panel) to β-actin. The ratios were determined based on the p-AMPK and AMPK band intensities quantified using Image Lab (Bio-Rad) and normalized to the loading control (β-actin). Data shown are mean SEM. *: p<0.05, **: p<0.01 using Wilcoxon rank sum test.

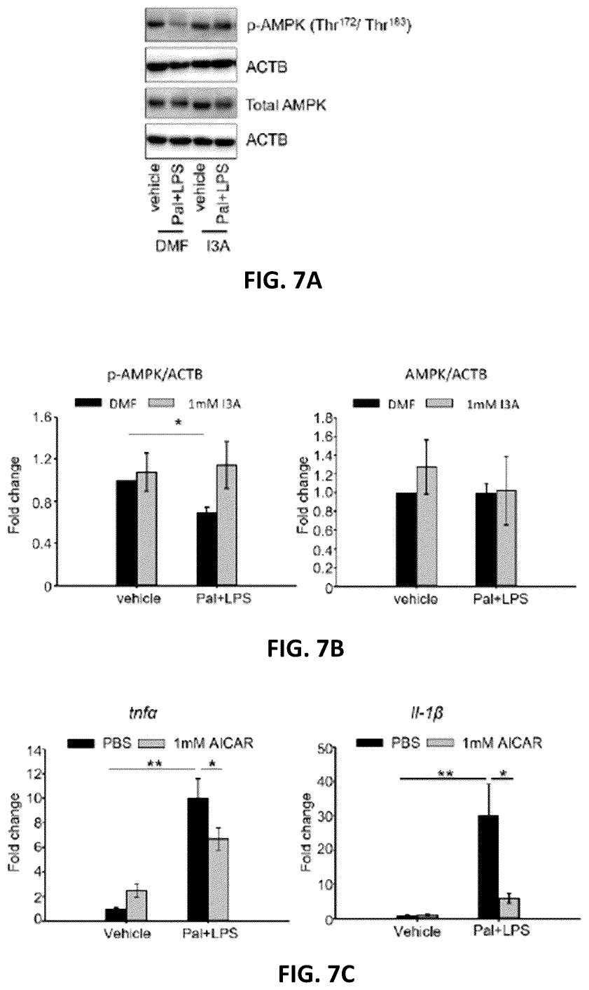

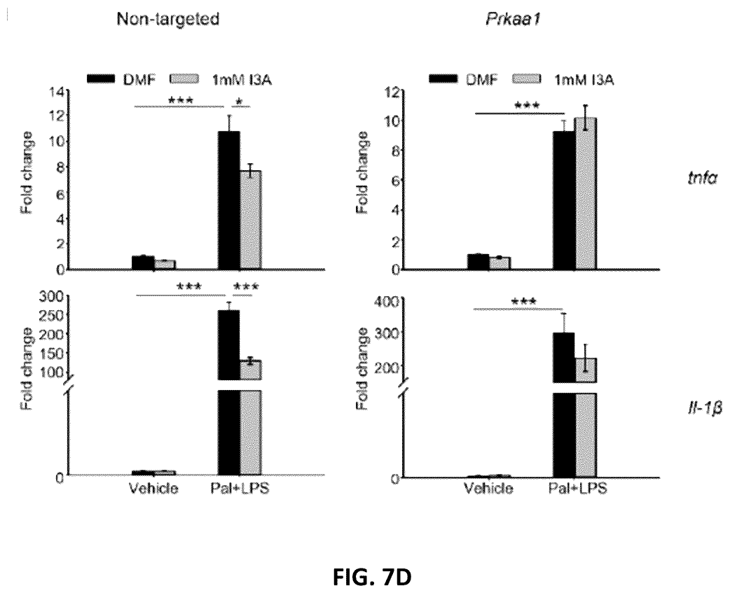

Using an in vitro model, a similar AMPK dependence of 3A's anti-inflammatory effect in murine macrophages was demonstrated. FIGS. 7A-7D demonstrate that I3A suppresses macrophage inflammation in vitro in an AMPK dependent manner. FIG. 7A is a photograph of a Western blot analysis of the expression levels of p-AMPK and total AMPK in RAW 264.7 macrophages pre-treated with either I3A or vehicle (DMF) control followed by stimulation with palmitate and LPS. FIG. 7B is a set of graphical representations of the fold-changes in p-AMPK and total AMPK. Fold-changes were calculated relative to the DMF and no palmitate and LPS stimulation condition. The band intensities were quantified and normalized to loading control (β-actin) by using Image Lab (Bio-Rad). FIG. 7C is a set of graphical representations of the expression levels of TNFα and IL-1β in RAW 264.7 cells treated with p-AMPK activator AICAR, followed by stimulation with palmitate and LPS. FIG. 7D is a set of graphical representations of the expression levels of TNFα (top row) and IL-1β (bottom row) in RAW 264.7 cells transduced with non-targeted control siRNA (left panels) or Prkaa1 siRNA (right panels), pre-treated with I3A, and then stimulated with palmitate and LPS. Data show are mean±SEM from three independent cultures with three biological replicates. *: p<0.05, **: p<0.01, ***: p<0.001 using Student's t-test. Whether AMPK signaling plays a role in mediating I3A's effects in hepatocytes in vivo and whether this is important relative to I3A's activation of the AhR warrants further investigation.

Oral administration of a microbiota derived tryptophan metabolite, I3A, in WD-fed animals alleviates diet-induced liver steatosis and inflammation, even when the animals were continued on WD. These hepatoprotective effects occurred without significant alterations in the gut microbiome composition and metabolome profiles, indicating that I3A acted directly on host cells. In vivo results show a correlation between AMPK phosphorylation and the efficacy of I3A, while in vitro studies with RAW macrophages show that AMPK mediates I3A's attenuation of pro-inflammatory cytokine expression.

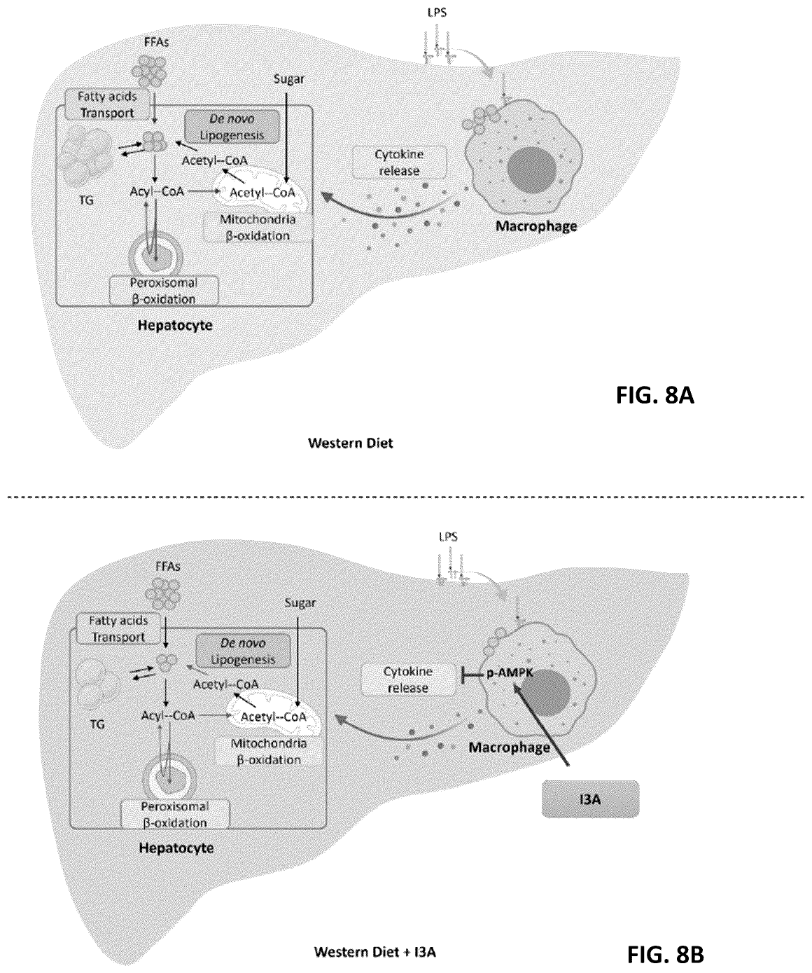

FIGS. 8A-8B are proposed models for the effects of I3A on hepatic lipid metabolism and inflammation. When mice are fed with a WD (FIG. 8A), TG and FFAs accumulate in the liver due to increased uptake of fatty acids. This also leads to increased β-oxidation in the mitochondria and peroxisomes. In liver macrophages, the increase in FFAs, possibly in conjunction with circulating endotoxins (e.g., LPS) stimulate production of inflammatory cytokines. When mice fed the WD are treated with I3A (FIG. 8B), both TG and FFAs decrease in the liver. Rather than impact fatty acid uptake, I3A treatment reduces de novo lipogenesis through a downregulation of Fasn, while also reducing both mitochondrial and peroxisomal β-oxidation. In macrophages, I3A attenuates fatty acid and LPS stimulated production of inflammatory cytokines through activation of AMPK. While additional studies are warranted to investigate the above-mentioned possibilities, the remarkable potency of I3A in alleviating steatosis and inflammation in a therapeutic model clearly demonstrates the potential for developing I3A as an inherently safe treatment option for NAFLD.

Gut microbiota derived tryptophan metabolites indole-3-acetate (I3A) and tryptamine (TA) were decreased in both cecum and liver of high-fat diet (HFD)-fed mice compared to low-fat diet (LFD)-fed control mice. In vitro, both I3A and TA attenuated the expression of inflammatory cytokines (TNFα, IL-β and Mcp-1) in macrophages exposed to palmitate and LPS. In hepatocytes, I3A significantly attenuated TNF-α and fatty acid induced inflammatory responses in an aryl-hydrocarbon receptor (AhR) dependent manner. I3A was evaluated for protection against NAFLD progression in vivo. Supplementation of I3A in drinking water alleviated liver steatosis and inflammation even when animals are continued on the NAFLD inducing diet, and that these effects correlate with a decrease in both lipogenesis and β-oxidation in the liver. The anti-inflammatory effects of I3A in macrophages were mediated by AMP-activated protein kinase (AMPK).

### B. Compositions and Methods for Treating Acute Respiratory Distress Syndrome (ARDS)

The compositions that contain immunomodulatory compounds provided herein can be safely administered to modulate the hyperinflammatory response in ARDS. Certain embodiments include the method of treatment of ARDS that develops in severely ill COVID-19 patients. The compounds can be tryptophan (Trp) derived microbial metabolites that attenuate the production of cytokines implicated in the hyperinflammation of COVID-19 patients. The tryptophan metabolite can include indole 3-acetate (I3A), a Trp metabolite that modulates the production of several inflammatory cytokines, or a derivative thereof. For example, administration of I3A through drinking water attenuates the production of inflammatory cytokines in mice. These cytokines include TNF-α, GM-CSF, IFN-γ, MCP-1, IL-1β, IL-6, IL-10, and IL-17A, which have been implicated in hyperinflammation of severely ill COVID-19 patients. The tryptophan metabolite can include tryptamine (TA) or a derivative thereof. The tryptophan metabolite can also include xanthurenic acid (XA) or a derivative thereof. These immunomodulatory compounds can attenuate the harmful excessive inflammatory response in ARDS, especially as occurring in severely ill COVID-19 patients.

Hyperinflammation drives severity of COVID-19. In COVID-19 patients who develop ARDS, circulating inflammatory cytokines rise early in the course of disease and remain elevated even after the virus clears from the lungs, whereas moderately ill patients show a progressive decrease in the cytokines following an initial increase, typically 6 to 10 days following symptom onset. Severely ill patients also show a sharp depletion in T cells and enrichment of monocytes and neutrophils, suggesting broad dysregulation of both adaptive and innate immune responses. This hyperinflammatory response (described as a cytokine storm) has been linked with poor prognosis for recovery and higher mortality. The cytokines implicated in this response are associated with all three major types of immunity, and include IL-1β, IL-2, IL-4, IL-6, IL-7, IL-10, IL-12, IL-13, IL-17A, M-CSF, G-CSF, GM-CSF, IP-10, IFN-γ, MCP-1, MIP 1-α, and TNF-α. The extent to which these cytokines increase in circulation of a COVID-19 patient depends on a number of factors, including the types of treatments received by the patient. However, a growing number of studies consistently report a positive association between disease severity and serum levels of IL-10, IL-6, and TNF-α. These observations have led to therapies targeting IL-6, but clinical trials have thus far yielded disappointing results. Blocking the inflammatory cytokine cascade early on in disease could compromise the body's ability to fight the inciting infection. Even if given later in the disease course, it could potentially place the patient at risk of secondary infection. Therefore, there is a pressing need to develop new options to safely and effectively treat the cytokine storm in COVID-19 patients.

Certain compounds that are Trp-derived gut microbiota metabolites can be administered as potent immunomodulatory compounds for treatment of ARDS. The GI tract harbors trillions of microorganisms belonging to several hundred species. This intestinal microbiota performs a diverse array of functions critical to the host's physiology. The intestine's mucosal immune system maintains a tolerance towards commensal microbes, while retaining the ability to recognize pathogens and pathogen-derived toxins. This allows the body to mount a measured immune response to potential antigens. Immunomodulatory metabolites produced by commensal microbes play a critical role in this immune homeostasis. In particular, certain Trp metabolites have been shown to exert anti-inflammatory effects in the GI tract. Indole, produced from Trp by the bacterial enzyme tryptophanase, is a AhR ligand capable of decreasing pro-inflammatory indicators and increasing tight junction resistance. In a murine model of NSAID enteropathy, indole administration decreased mucosal inflammation and injury. Administration of indole-3-aldehyde, another microbial Trp metabolite, induced IL-22 production from natural killer (NK) cells and protected against mucosal inflammation in murine models of colitis and fungal infection. Patients with inflammatory bowel disease show higher Trp levels in their stool, reduced serum IL-22 levels, and depletion of microbially derived, AhR-active Trp metabolites, including I3A. Taken together, these findings show that gut microbial Trp metabolites have immunomodulatory activity that protects against intestinal inflammation.

An untargeted analysis of serum metabolites in COVID-19 patients detected significant increases in kynurenine and kynurenate in severely ill patients compared to healthy controls, whereas the increases were not observed in moderately ill patients. Moreover, Trp levels were lower in COVID-19 patients compared to healthy controls. Similar trends, i.e., increase in kynurenine and decrease in Trp, along with a significant positive correlation between kynurenine and serum IL-6 levels in COVID-19 patients, have been reported.

In certain embodiments, I3A or TA or XA are administered to modulate the cytokine storm in COVID-19 by regulation of the NLRP3 (NOD-, LRR- and pyrin domain-containing protein 3) inflammasome through activation of the aryl hydrocarbon receptor (AhR). Activation of the inflammasome in alveolar and monocyte-derived macrophages (possibly due to overproduction of TNF-α) has been observed in other coronavirus infections. Moreover, transduction of murine bone marrow derived macrophages with SARS-Cov-2 3a, a viroporin, resulted in NLRP3 inflammasome activation and secretion of IL-1β. In these embodiments, I3A or TA activate AhR, resulting in inhibition of NLRP3 inflammasome in cytokine/antigen stimulated macrophages (and epithelial cells), and attenuation of proinflammatory signaling.

In certain embodiments, I3A or TA or XA are administered to directly engage AhR-dependent or independent cytokine pathways to regulate immune homeostasis in the epithelial cells of the lungs, the intestine, or other organs. I3A, TA, and XA are microbiota-dependent metabolites capable of activating the AhR. Targeted LC-MS experiments comparing cecum and fecal samples from conventionally raised (CONV-R) and germ-free (GF) mice showed significant depletion of I3A, TA, and XA (along with indole and 5-hydroxy-L-tryptophan) in GF mice, confirming that the quantities of these Trp-derived metabolites depend on the gut microbiota. Cell-based reporter assays showed that both I3A and TA dose-dependently activate the AhR to an extent that is comparable to other endogenous Trp-derived ligands (e.g., kynurenine). Furthermore, high-fat diet (HFD)-induced steatosis reduced the levels of all three metabolites in cecum, serum, and liver of mice. Treatment with I3A or TA dose-dependently attenuated the production of TNF-α, IL-1β, and MCP-1 in palmitate and LPS stimulated macrophages. In cultured hepatocytes loaded with lipid droplets (mimicking steatosis), treatment with I3A abrogated TNF-α induced production of saturated free fatty acids. Inhibition experiments with a selective antagonist showed that this effect was AhR dependent. I3A attenuates expression of inflammatory cytokines in diet-induced steatohepatitis.

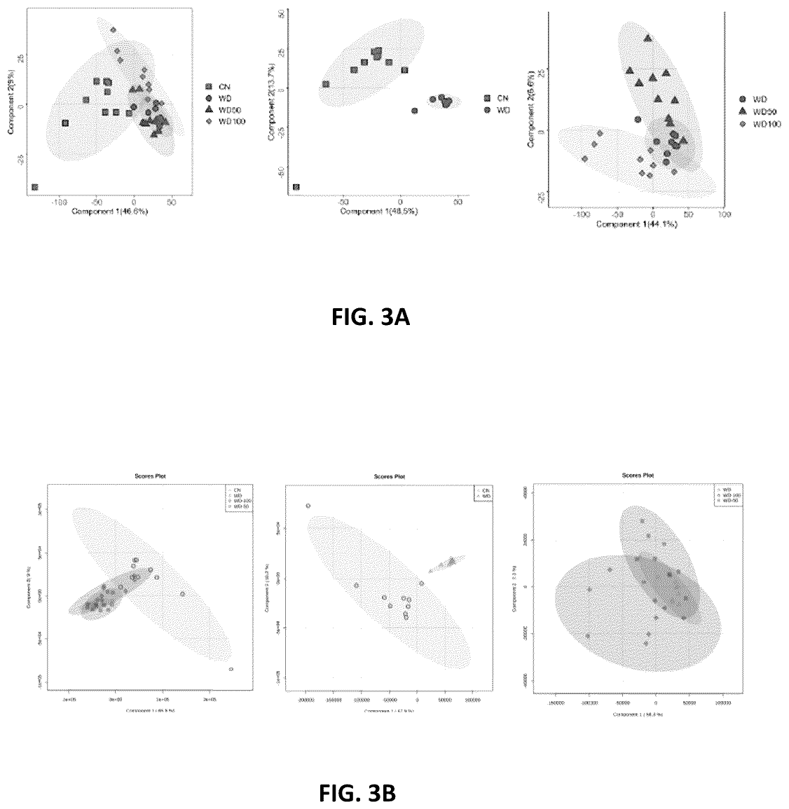

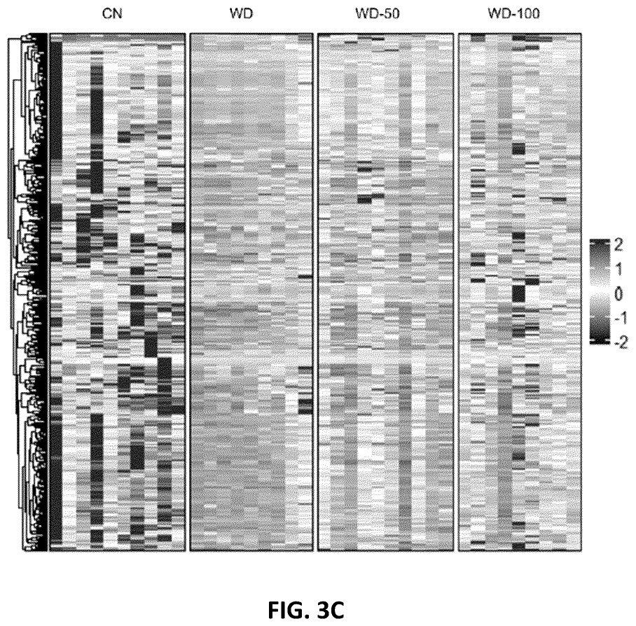

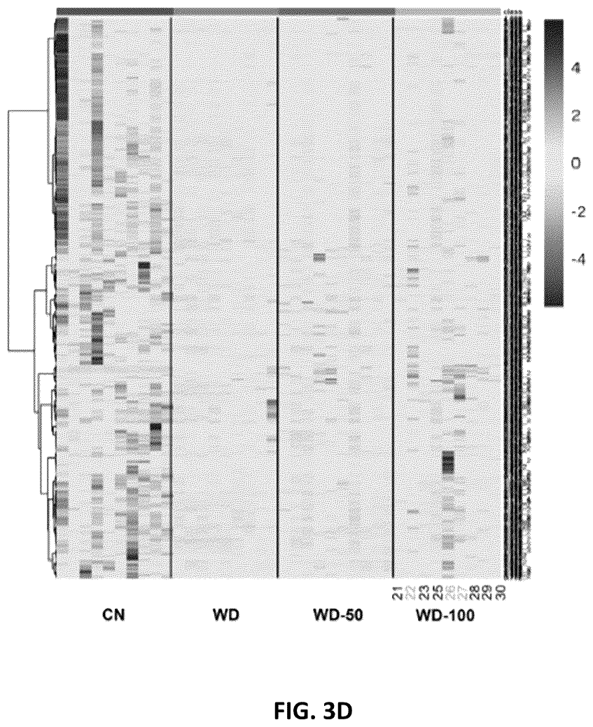

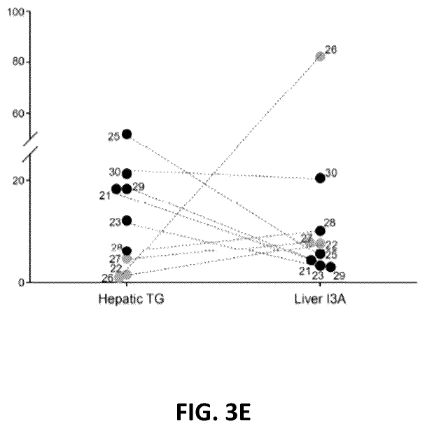

I3A was demonstrated to exert anti-inflammatory effects in vivo using a murine model of non-alcoholic steatohepatitis (NASH). After 16 weeks on a Western (high-fat/high-sugar) diet (WD), B6/129 mice showed macro- and micro-vesicular steatosis and extensive immune cell infiltration in livers. Administration of I3A (up to 100 mg/kg body wt.) in drinking water dose-dependently protected against these injuries. Moreover, I3A administration dose-dependently attenuated the production of type 1, 2 and 3 cytokines, including TNF-α, MCP-1, IL-1β, IL-6, IL-17A, IL-23, IFN-β, IFN-γ, IL-27, IL-1 α, GM-CSF, and IL-1β (FIG. 3).

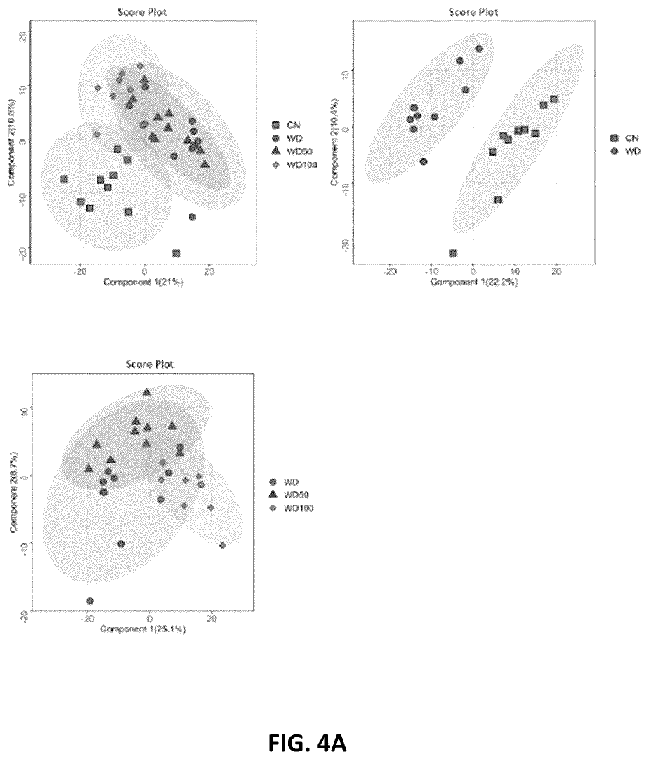

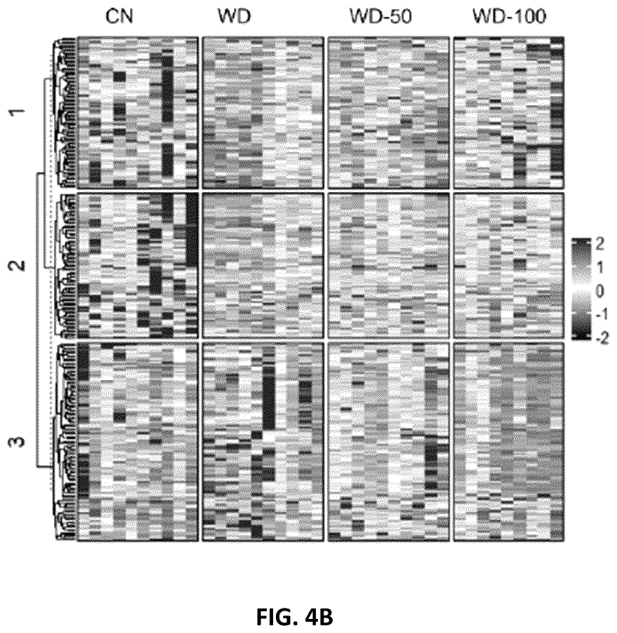

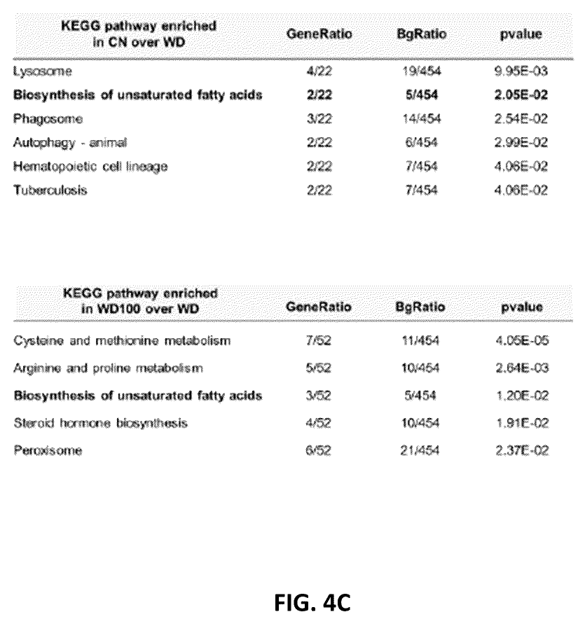

FIGS. 1A-1F demonstrate that oral supplementation of I3A through drinking water alleviates liver steatosis and inflammation in a Western Diet mouse model. FIG. 1A is a graphical representation of I3A levels in the feces with Western Diet (WD) before I3A supplementation (left panel) and after 8 weeks of I3A supplementation (right panel). FIG. 1B is a graphical representation of I3A levels in the liver after 8 weeks of I3A supplementation. FIG. 1C is a graphical representation of the serum ALT levels before I3A supplementation (left panel) and after 8 weeks of I3A supplementation (right panel). FIG. 1D is a graphical representation of the triglyceride levels in the liver 8 weeks of I3A supplementation. CN: control mice; WD: WD mice at the start of I3A supplementation; WD-50: WD mice with 50 mg/kg body weight of I3A supplementation through drinking water, WD-100: WD mice with 100 mg/kg body weight if I3A supplementation through drinking water. FIG. 1E is a set of photographic images of H&E staining of liver sections. FIG. 1F is a graphical representation of liver steatosis, hepatocyte ballooning, and lobular inflammation scored from 3 stained sections per animal. *: p<0.05, **: p<0.01, and ***: p<0.005. FIG. 2 is a set of graphical representations of the primary, secondary, and conjugated primary bile acid levels in the liver with I3A supplementation. CN: control mice, WD-50: WD mice with 50 mg/kg body weight through drinking water, WD-100: WD mice with 100 mg/kg body weight through drinking water. *: p<0.05, **: p<0.01, and ***: p<0.005. FIG. 3 is a set of graphical representations of the effect of I3A on inflammatory cytokine levels in livers of male B6/129 mice (n=10 mice per group). Animals were fed either a control (CN) or Western diet (WD). A randomly selected subset of WD mice were given an average daily dose of 50 (WD-50) or 100 (WD-100) mg of I3A per kg body wt. Cytokines were measured using a bead-based multiplex assay in homogenized, lyophilized, and defatted liver samples. Dots and bars show individual liver values and means. *: p<0.05, **: p<0.01, ***: p<0.001 by Wilcoxon rank-sum test. Principal component analysis (PCA) is used to visualize high-dimensional metabolomic data in a two-dimensional subspace. FIG. 4A is a set of principal coordinate analysis of liver metabolome with I3A supplementation. FIG. 4B is a heat map showing hierarchical clustering of the different groups. FIG. 4C is a correlation between TG and I3A levels in livers from the WD-100 group. CN: control mice, WD-50: WD mice with 50 mg/kg body weight through drinking water, WD-100: WD mice with 100 mg/kg body weight through drinking water. Taken together, these preliminary results demonstrate that I3A and TA are potent immunomodulatory compounds.

### C. Dosage, Route, and Timing of Administration

A subject (for example a human subject having an inflammation-associated disease, such as a liver disorder or ARDS) can be administered a therapeutic amount of pharmaceutical compositions comprising an immunomodulatory compound (such as a tryptophan metabolite or a derivative thereof, such as indole 3-acetate, tryptamine, xanthurenic acid, or a derivative of any thereof) in accordance with any dosage, route, and timing that suits the clinical needs of the subject.

For example, in some embodiments, the composition is administered to the subject orally. In other embodiments, the composition is administered to the subject via parenteral route, such as, intravenously, enterally, by inhalation, intranasally, intratracheally, topically, subcutaneously, intradermally, or intrathecally. For example, the composition can be administered orally as a medication or a dietary supplement in a powder, capsule, gel, paste, pouch, tablet, or oils; in combinations with food items or alone; as a suppository in lubricants or sachets; topically; or via other administration routes. In some embodiments, the composition is formulated for delivery to a target organ, such as to the liver or to the lung. For example, the composition can be formulated as a powder or a capsule for oral administration and delivery to the liver. The composition can be formulated as a liquid for inhalation or intranasal administration and delivery to the lung.

The immunomodulatory compound can be formulated in a composition with a carrier or other agents. As used herein, the term “carrier” refers to an inert compound that is compatible with any other ingredients in the formulation and is not deleterious to the active compound (such as the immunomodulatory compound) or a subject that the formulation is administered thereto. Suitable carriers can be added to improve recovery, efficacy, or physical properties and/or to aid in packaging and administration. Such carriers may be added individually or in combination. Non-limiting examples of carriers include proteins, carbohydrates, fats, enzymes, vitamins, immune modulators, oligosaccharides, milk replacers, minerals, amino acids, coccidiostats, and acid-based products. Common carriers include cellulose, sugar, glucose, lactose, whey powder, and rice hulls.

In some embodiments, the administration of the composition is repeated, for example, hourly, twice per day, daily, twice per week, weekly, biweekly, or monthly. In some embodiments, the administration is for a prescribed time period, for example for one month, two months, three months, four months, five months, six months, one year, longer, or indefinitely. After an initial treatment, the subsequent treatments can be administered less frequently relative to the initial treatment.

In some embodiments, the composition is administered in one dose, or in two or more doses. In some embodiments, the number, frequency, or amount of subsequent doses is dependent on the achievement of a desired therapeutic effect. In some embodiments, the composition is administered to a subject at the frequency and amount required to achieve a therapeutic effect. In some embodiments, the subject can be monitored for desired therapeutic effects and unwanted side effects associated with administration of the composition.

### EXAMPLES

The following examples are presented to illustrate select aspects of the various embodiments, including compositions and methods of treatment of liver disorders, including steatosis, steatohepatitis, and other inflammation-induced diseases.

### Example 1: Oral Administration of I3A Alleviates Diet Induced Hepatic Steatosis and Inflammation

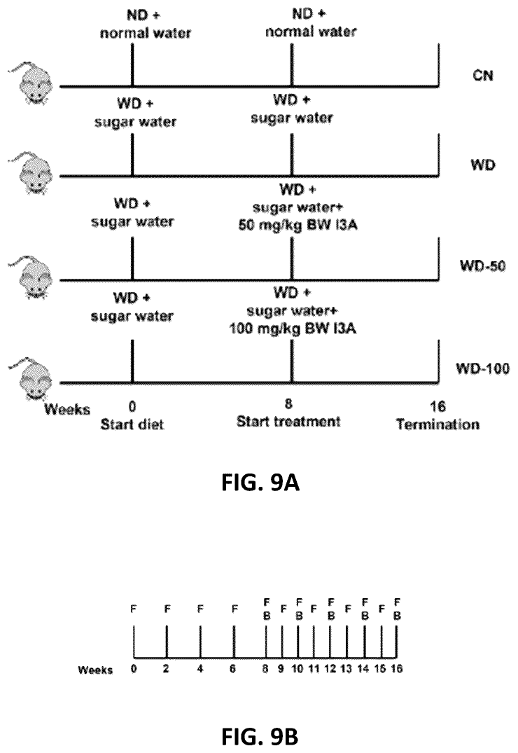

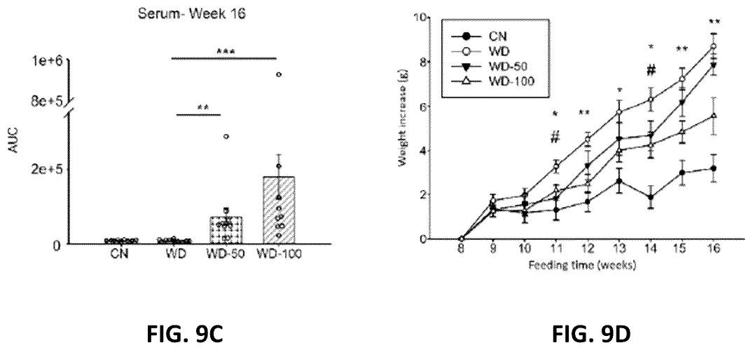

A mouse model of diet-induced fatty liver disease was used to investigate the effect of I3A administration through drinking water (FIG. 9A) on liver steatosis and inflammation. The levels of I3A were significantly reduced in fecal material (50% and 64% decrease at week 8 and 16, respectively) and the liver (70% decrease at week 16) of Western diet (WD) fed mice compared to control mice (CN) fed a low-fat diet. Mice that were given WD and sugar water (SW) containing I3A showed increased I3A levels in fecal material, the liver and serum compared to mice given the same WD and SW without I3A (FIGS. 9A-9C). The impact of I3A administration on WD induced features of NAFLD was evaluated. FIG. 9C is a graphical representation of the quantification of I3A in serum. Compared to CN, WD fed animals had elevated serum alanine aminotransferase (ALT) levels, indicating liver injury. Treatment of WD fed animals with the high dose of I3A (WD-100) significantly reduced serum ALT levels two weeks after beginning the I3A treatment (week 10-time point, FIG. 1C) and beyond. Additionally, I3A treatment reduced liver TG in a dose-dependent manner at week 16 (FIG. 1D). Scoring of H&E-stained liver sections indicated that the I3A treatment improved liver steatosis, hepatocyte ballooning, and lobular inflammation in a dose-dependent manner (FIGS. 1E and F). Treatment with I3A also slowed the weight increase of WD fed mice (FIG. 9D). FIG. 9D is a graphical representation of the body weight increase of the four groups, normalized to week 8 body weight when I3A administration was started. Data shown are mean±SEM. *: p<0.05, **: p<0.01, WD-100 group compared to WD group using Wilcoxon rank sum test. #: p<0.05, WD-50 group compared to WD group using Wilcoxon rank sum test.

I3A treatment modulated the levels of inflammatory cytokines in WD-fed animals. All pro-inflammatory cytokines in the panel such as TNFα, IL-6 and Mcp-1 etc. were significantly elevated in livers of WD mice compared to CN (FIG. 2A and FIG. 10A). Treatment with I3A reduced the expression of these cytokines in a dose-dependent manner. Interestingly, WD upregulated IL-10, considered an anti-inflammatory cytokine, and I3A treatment downregulated this cytokine (FIG. 10A). Taken together, these results demonstrate that I3A, provided via drinking water, attenuates diet-induced hepatic steatosis, cellular injury, and inflammation.

### Example 2: I3A Reverses Diet Induced Alterations in Liver Bile Acids and Free Fatty Acids

As studies in mice and humans showed that NAFLD is associated with alterations in liver bile acids, the levels of 13 major bile acid species were quantified in the livers of the different treatment groups (Table 1). Mice in the WD group showed significantly reduced liver total bile acids and increased primary bile acids (FIG. 2B). The WD-100 group showed the opposite trend, whereas the WD-50 group did not show any significant changes in liver bile acid profile compared to the WD group. The concentrations of nine major free fatty acids were also determined (FFAs, Table 2). In the liver, WD significantly increased the total concentration of these FFAs by 2.7-fold compared to CN (FIG. 10B). Treatment with I3A had no significant effect, although the total FFA concentration trended lower in the WD-100 group (36% reduction, p=0.11, FIG. 10B). The total FFA concentrations in serum were similar across all four groups (FIG. 10B).

TABLE 1
LC-MS parameters for bile acid analysis

| | RT | | Precursor | Product | CE | RF Lens |
|---|---|---|---|---|---|---|
| Bile acid | (min) | Polarity | (m/z) | (m/z) | (V) | (V) |
| Muricholic acid | 1.39 | Negative | 407.3 | 371.3 | 31.7 | 249 |
| Tauromuricholic acid | 0.9 | Negative | 514.5 | 79.9 | 55 | 185 |
| Tauro-β-muricholic acid | 1.87 | Negative | 514.5 | 80 | 55 | 248 |
| Taurohyodeoxycholic acid | 1.58 | Negative | 498.6 | 79.9 | 55 | 137 |
| Murideoxycholic acid | 2.54 | Negative | 391.5 | 327.3 | 34.3 | 170 |
| Glycocholic acid | 1.76 | Negative | 464.5 | 74 | 36.8 | 111 |
| Taurocholic acid | 1.87 | Negative | 514.5 | 79.9 | 55 | 137 |
| Ursodeoxycholic acid | 2.2 | Negative | 391.4 | 355.2 | 32.4 | 178 |
| Cholic acid | 2.64 | Negative | 407.8 | 290.3 | 37.5 | 131 |
| Hyodeoxycholic acid | 2.54 | Negative | 391.5 | 353.2 | 35.7 | 173 |
| Taurodeoxycholic acid | 3.05 | Negative | 498.4 | 79.9 | 54.6 | 198 |
| Deoxycholic acid | 4.83 | Negative | 391.4 | 327.2 | 34.6 | 146 |
| Chenodeoxycholic acid | 4.6 | Negative | 391.4 | 355.1 | 34.8 | 154 |

TABLE 2
LC-MS parameters for free fatty acid analysis

| | | | | | |
|---|---|---|---|---|---|
| | RT | [M − H]− | TOF MS² | CE | |
| Free fatty acids | (min) | (m/z) | (m/z) | (V) | DE |

Light Experiment for quantification

| | | | | | |
|---|---|---|---|---|---|
| Lauric acid | 1.94 | 199.17 | 199.17 | −8 | −100 |
| Myristic acid | 3.28 | 227.2 | 227.2 | −15 | −150 |
| Palmitic acid | 6.02 | 255.23 | 255.23 | −15 | −150 |
| Palmitoleic acid | 3.91 | 253.22 | 253.22 | −15 | −150 |
| Stearic acid | 10.81 | 283.26 | 283.26 | −15 | −150 |
| Oleic acid | 7.01 | 281.25 | 281.25 | −15 | −150 |
| Linoleic acid | 4.79 | 279.22 | 279.22 | −17 | −150 |
| Linolenic acid | 3.38 | 277.22 | 277.22 | −15 | −150 |
| Arachidonic acid | 4.57 | 303.23 | 303.23 | −10 | −175 |

Heavy Experiment for confirmation

| | | | | | |
|---|---|---|---|---|---|
| Lauric acid | 1.94 | 199.17 | 199.17 | −21 | −100 |
| Myristic acid | 3.28 | 227.20 | 227.20 | −23 | −150 |
| Palmitic acid | 6.02 | 255.23 | 255.23 | −26 | −150 |
| Palmitoleic acid | 3.91 | 253.22 | 253.22 | −25 | −150 |
| Stearic acid | 10.81 | 283.26 | 283.26 | −25 | −150 |
| Oleic acid | 7.01 | 281.25 | 281.25 | −26 | −150 |
| Linoleic acid | 4.79 | 279.22 | 279.22 | −25 | −150 |
| Linolenic acid | 3.38 | 277.22 | 277.22 | −23 | −150 |
| Arachidonic acid | 4.57 | 303.23 | 303.23 | −20 | −175 |

### Example 3: I3A Administration does not Significantly Alter the Fecal Microbial Community as Well as Fecal Metabolome

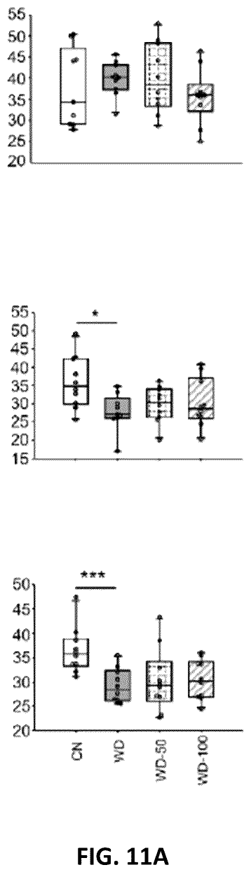

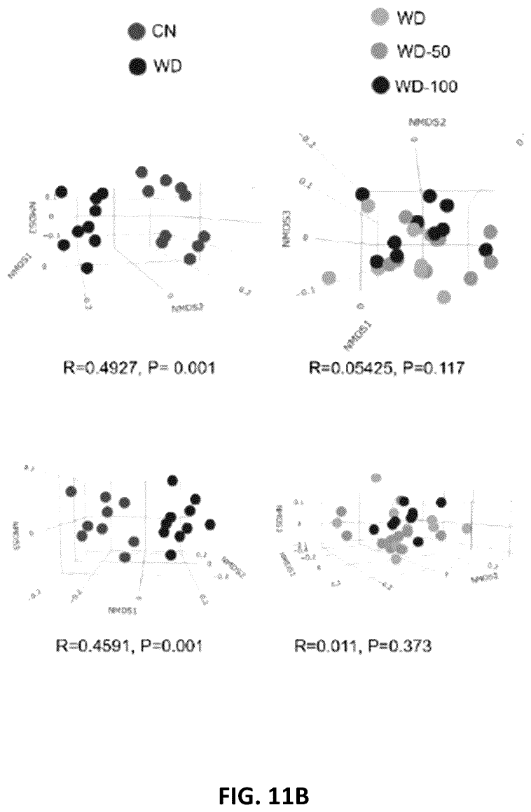

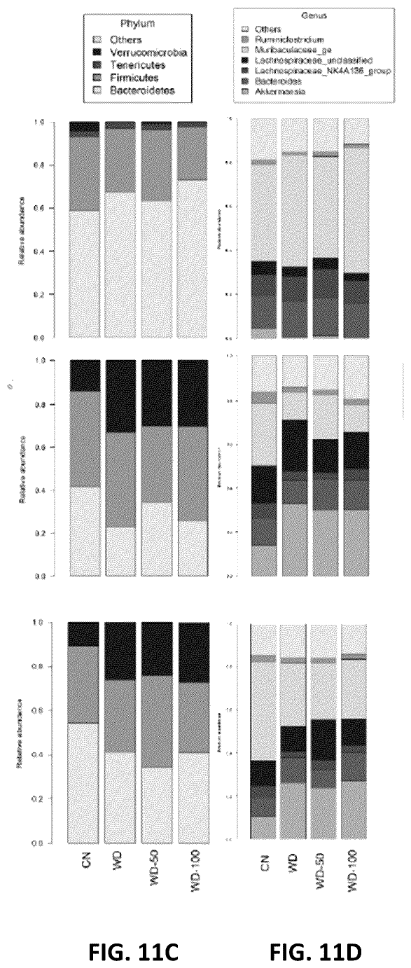

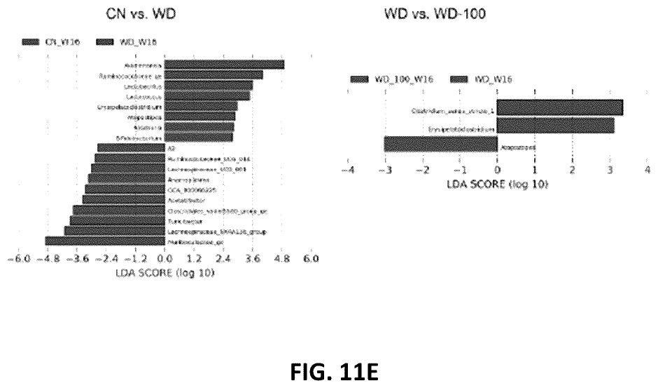

Next, whether the hepatoprotective effects of I3A were due to a modification of the gut microbiome was investigated. The fecal microbial communities at week 8 of WD (prior to I3A treatment) and week 16 (at termination) were analyzed using 16S rRNA sequencing. Fecal microbiome of WD fed mice showed reduced α-diversity compared to CN, which was not further altered upon I3A treatment (FIG. 11A). Similarity analysis using the Bray-Curtis dissimilarity metric showed that the microbiome compositions of CN and WD-fed mice were significantly different (FIG. 11B). The fecal microbiome compositions of WD-fed mice did not change significantly upon I3A treatment (FIG. 11B). Analysis of fecal microbiota taxonomic profiles at the phylum and genus level (FIGS. 11C and 11D) showed major shifts in bacterial abundance of fecal microbiota from WD-fed mice relative to CN. At week 16, the relative abundance of phylum Verrucomicrobia increased, whereas Bateroidetes decreased. LEfSe detected 18 genera showing significant differences in relative abundance between the WD group and CN, including an expansion of Akkermansia and reduction of Muribaculaceae. In contrast, only 3 genera showed significant shifts in WD fed mice upon I3A treatment (FIG. 11E).

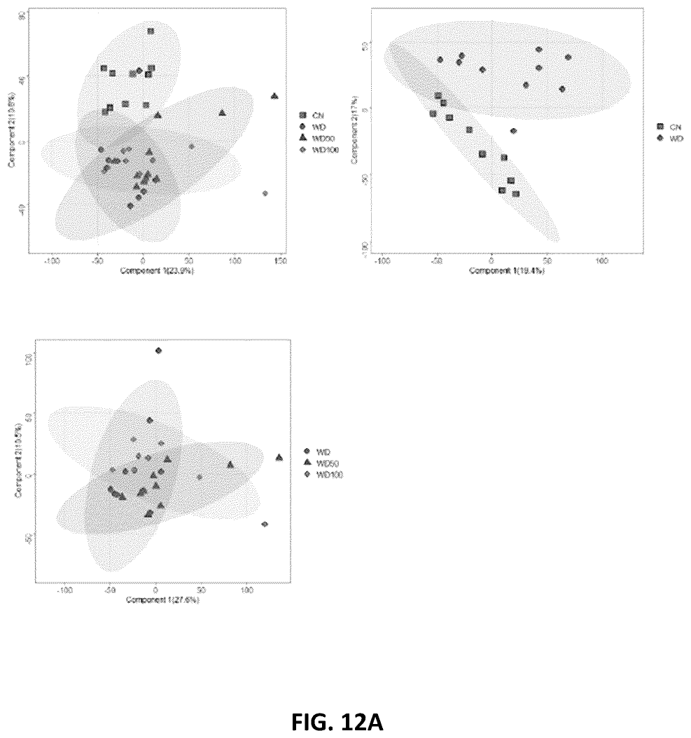

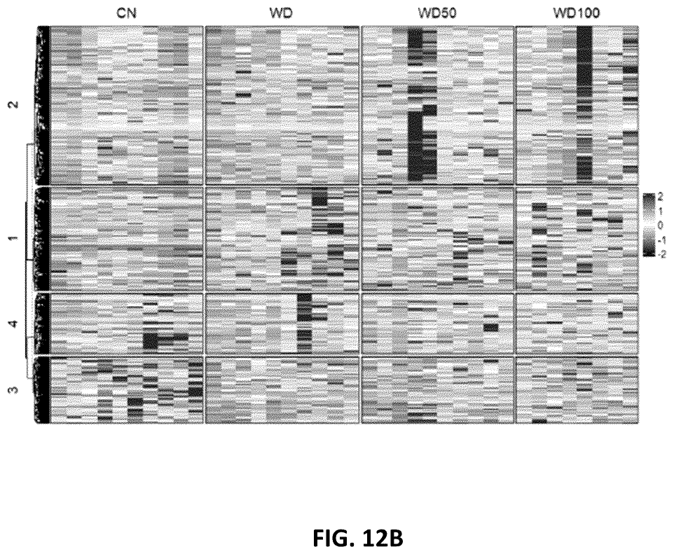

Whether administration of I3A altered the metabolic profile of the gut microbiota was also investigated. Principal component analysis (PCA) of untargeted LC-MS data showed that the fecal metabolome of WD fed mice was significantly different from CN, whereas the metabolomes of WD-100 and WD groups largely overlapped (FIG. 12A). Although permutational multivariate analysis of variance (PERMANOVA) suggested a modest difference in the variance of metabolite levels between WD-50 and WD groups, Hotelling's T2 test found that the means (centroids) were not significantly different. Clustering analysis using the k-means showed that fecal metabolites could be assigned into 4 groups (FIG. 12B). No clusters were observed that showed a reversal of WD induced changes in fecal metabolome by I3A treatment. Taken together, these results indicate that treatment with I3A had minimal effects on the fecal microbiota composition as well as metabolome of WD fed mice.

### Example 4: I3A Administration Partially Reverses Diet Induced Metabolome Alterations in the Liver

The impact of I3A treatment on the liver metabolome using untargeted LC-MS was investigated. Of the 3,463 distinct features detected across all samples, 2,883 were significantly increased or decreased in at least one group to group comparison. Annotation of the data putatively identified 516 of the significant features. Projections of the metabolite data onto latent variables from PLS-DA showed that the liver metabolome of WD-fed mice was significantly different (p<10-5, Hotelling's T2) from CN (FIGS. 3A, 3B). The low dose (WD-50) group overlapped completely with the WD group (p=0.26), whereas the high dose (WD-100) group only partially overlapped with both CN and the WD group and was significantly different (p<0.005 and p<0.0005, respectively) from both groups (FIGS. 3A, 3B). To determine the major trends associated with WD feeding and I3A treatment, a hierarchical clustering analysis of the 516 significant and annotated features was performed. Most of these features were reduced in WD-fed mice compared to the CN mice. Treatment with the high dose of I3A generally reversed these changes (FIGS. 3C, 3D). However, the treatment response varied from animal to animal. Within the WD-100 group, mice with the highest I3A concentration and lowest hepatic TG concentration had metabolic profiles that were more similar (based on correlation distance) to CN than the WD group (FIG. 3E). These data suggest that administration of I3A partially reverses the WD-induced alterations in the liver metabolome, which correlates with amelioration of steatosis.

### Example 5: I3A Administration Partially Reverses Diet Induced Proteome Alterations in the Liver

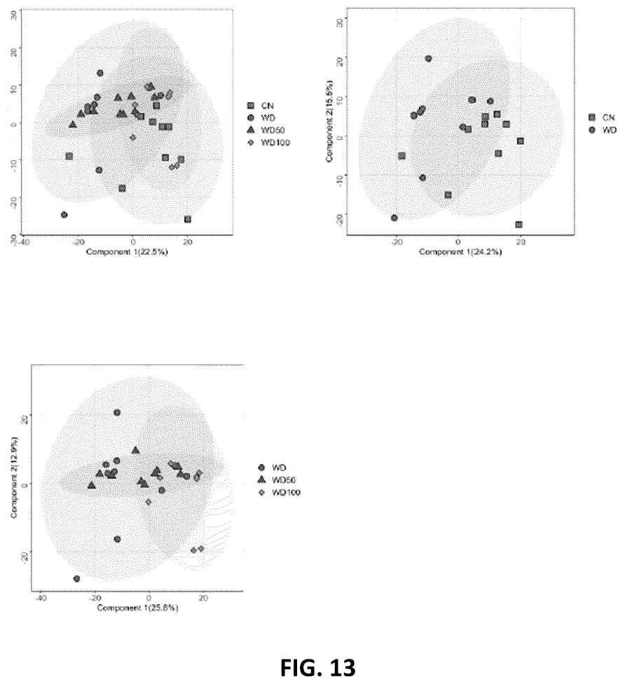

Whether I3A changed the expression levels of liver proteins altered by the WD was also evaluated. Latent variable projections from PLS-DA on liver proteomics data (FIG. 4A) showed that the WD group had a protein abundance profile significantly different from CN (p<10-6, Hotelling's T2). The effect of I3A was dose-dependent; whereas the WD-50 group was not significantly different from the WD group, the WD-100 group was significantly different from the WD group (p<0.001). The PLS-DA projections for WD-100 were closer to CN than WD, but still significantly different from CN. Principal component analysis (PCA) of the proteomics data showed similar trends (FIG. 13).

Clustering and silhouette analysis on significant (differentially abundant) proteins with VIP scores >1.2 in the PLS-DA projections identified three clusters of proteins based on their abundance profiles. Proteins in cluster 1 had lower abundance in the WD group compared to CN. This trend was reversed in the WD-100 group (FIG. 4B). Proteins in cluster 2 had lower abundance in the WD group compared to CN, but this trend was not reversed in the WD-100 group. Proteins in cluster 3 had higher abundance in the WD group compared to CN. These proteins had similar abundance in the WD-100 group and CN. The protein abundance profiles were similar between WD-50 and WD groups in all three clusters. These results suggest that the I3A treatment partially reversed the diet-induced alterations in the liver proteome in a dose-dependent manner, similar to the trend observed for the liver metabolome.

An enrichment analysis was performed to identify liver biochemical pathways significantly altered by diet and I3A treatment. In total, 454 quantified proteins were associated with 276 KEGG pathways. Of these, 22 proteins were differentially abundant in WD mice compared to CN. These proteins were associated with 6 significantly enriched pathways (FIG. 4C). A comparison of the WD-100 and WD groups identified 52 differentially abundant proteins that were associated with 5 significantly enriched pathways. One metabolic pathway, biosynthesis of unsaturated fatty acids, was common to both sets of enriched pathways.

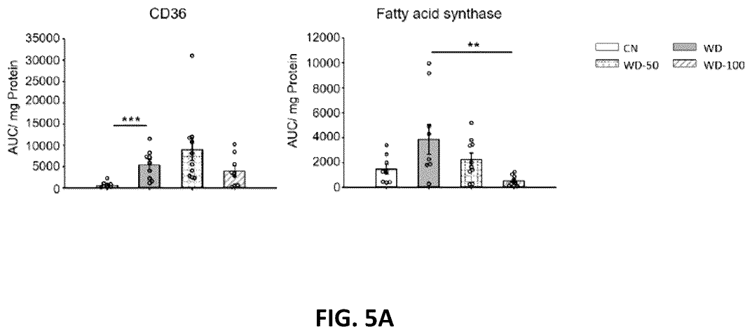

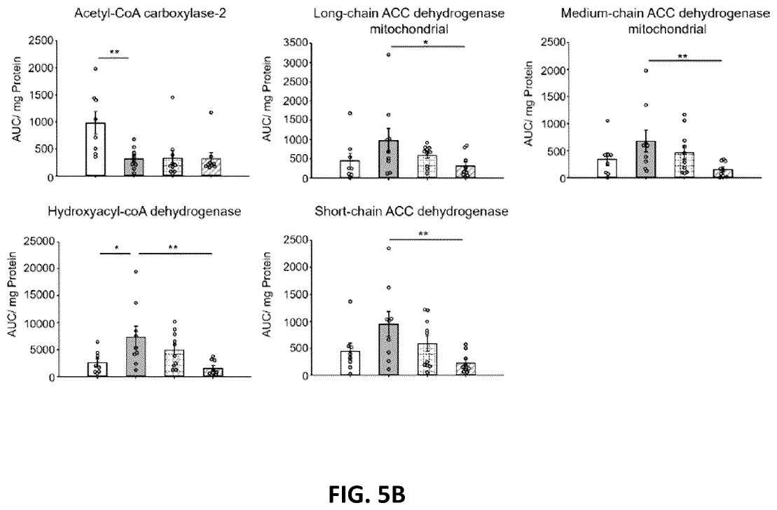

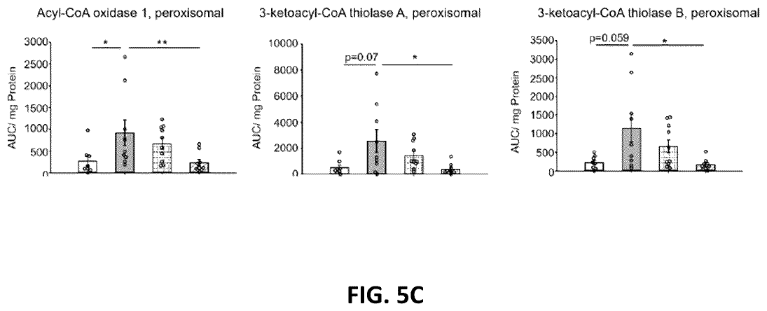

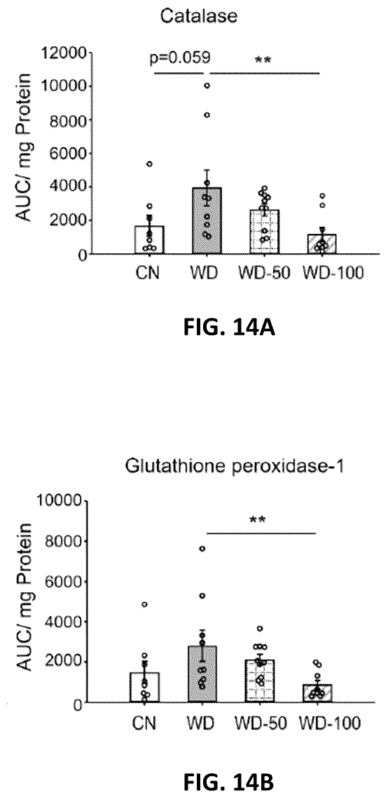

Targeted proteomics was used to quantify fatty acid metabolism enzymes identified in the untargeted proteomics data (FIGS. 5A-5C). Platelet glycoprotein 4 (CD36), a major fatty acid uptake protein, was elevated in the WD group compared to CN. The abundance of CD36 was decreased, but not significantly, in the WD-100 group compared to WD (FIG. 5A). The abundance of fatty acid synthase (Fasn) was not significantly different between the WD group and CN but was decreased in the WD-100 group compared to the WD group (FIG. 5A). Acetyl-CoA carboxylase-2 (Acab), which regulates mitochondrial β-oxidation, was decreased in the WD group compared to CN, but was not significantly different between the WD-100 group compared to WD (FIG. 5B). Enzymes catalyzing mitochondrial β-oxidation, including long-chain acyl-CoA dehydrogenase (Acadl), medium-chain acyl-CoA dehydrogenase (Acadm), short-chain acyl-CoA dehydrogenase (Acads) and hydroxyacyl-CoA dehydrogenase (Hadh) trended higher in the WD group compared to CN, although only Hadh showed a statistically significant difference (FIG. 5B). All four enzymes were significantly decreased in the WD-100 group compared to WD. A similar trend was observed for peroxisomal β-oxidation enzymes acyl-CoA oxidase 1 (Acox1) and peroxisomal 3-ketoacyl-CoA thiolases (Acaala and Acaalb, FIG. 5C). These results suggest that WD feeding led to increased cellular uptake, transport into mitochondria, and β-oxidation of fatty acids, while administration of I3A reduced fatty acid synthesis and β-oxidation. As β-oxidation generates reactive oxygen species (ROS), we also performed a targeted analysis of two antioxidant enzymes detected by the untargeted proteomics experiments, catalase (CatB) and glutathione peroxidase 1 (Gpx1). Both enzymes showed a dose-dependent decrease with I3A treatment (FIGS. 14A-14B).

### Example 6: I3A Suppresses Macrophage Inflammation in an AMPK Dependent, but not AhR Dependent Manner

Given that AMPK is a well-known signaling mediator in lipid metabolism and inflammation, and has been shown to play a role in the progression of NAFLD, whether the levels of AMPK and p-AMPK were altered in the different treatment groups was investigated. Both p-AMPK and AMPK were significantly downregulated in livers of the WD group compared to CN (FIG. 6A). Administration of I3A reversed this effect in a dose-dependent manner and increased both AMPK as well as p-AMPK (FIG. 6B).

The role of AMPK in RAW 264.7 macrophages in vitro was tested. Palmitate and LPS treatment significantly reduced p-AMPK levels by 50% compared to vehicle control but did not affect total AMPK (FIGS. 7A and 7B). Treatment with I3A upregulated p-AMPK to baseline levels without altering total AMPK (FIG. 7B). Palmitate and LPS also led to an increase in TNFα and IL-1β expression (FIG. 7C), while activation of AMPK with AICAR attenuated the palmitate and LPS stimulated increase in TNFα and IL-1β expression by 30% and 80%, respectively (FIG. 7C). These results suggest that upregulation of p-AMPK modulates the expression of TNFα and IL-1β in RAW 264.7 macrophages.

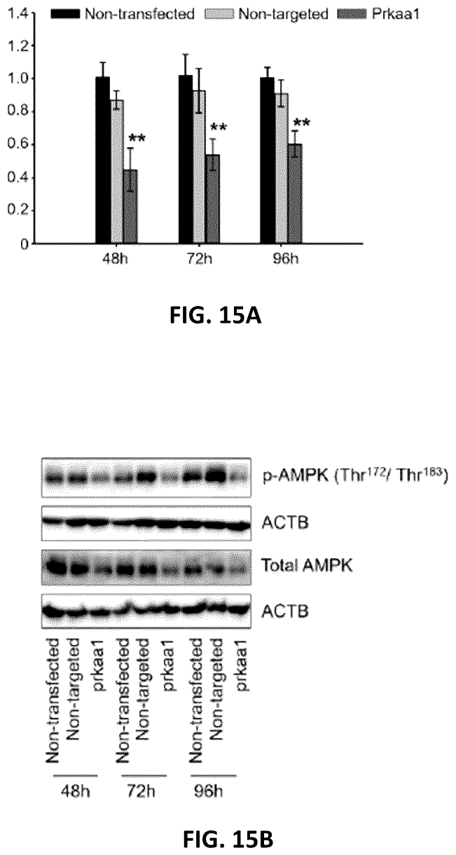

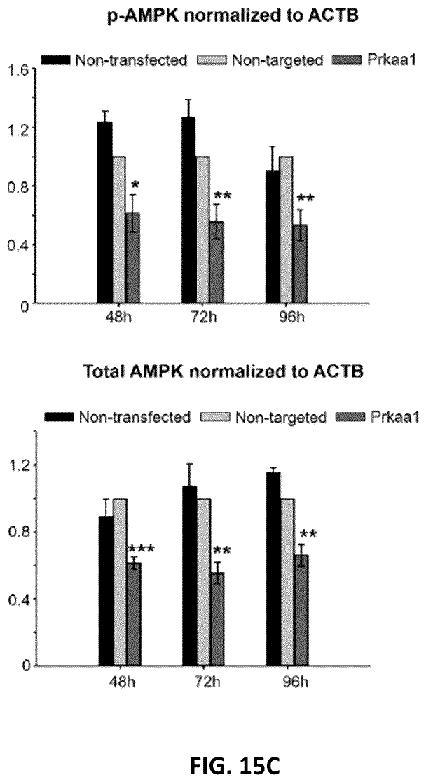

Whether the anti-inflammatory effect of I3A in RAW macrophages depends on AMPK activation was evaluated. Certain siRNAs were used to knock down prkaa1, the gene encoding AMPKα1, the main form of AMPKα in murine macrophages, and measured its effect on pro-inflammatory cytokine expression. The knockdown efficiency was ˜50% for mRNA (FIG. 15A), ˜40-50% for AMPK protein, and ˜50-60% for p-AMPK (FIGS. 15B and 15C), and the knockdown was stable for up to 96 h at both mRNA and protein levels. In cells transfected with the non-targeted control siRNA (FIG. 7D, left panel), palmitate and LPS increased TNFα and IL-1β expression by ˜10- and 250-fold, respectively, and I3A significantly downregulated this by ˜30% and 50% for TNFα and IL-1β, respectively, relative to the DMF control. In contrast, I3A did not significantly modulate TNFα expression in prkaa1 siRNA transfected cells stimulated with palmitate and LPS, and only reduced IL-1β expression by ˜20% (FIG. 7D, right panel) relative to the DMF control. These results suggest that I3A's anti-inflammatory effect in RAW 264.7 macrophages depend on AMPK activation.

The metabolic effects of I3A in hepatocytes depend on the AhR. Therefore, the role of AhR in I3A's effect in macrophages was investigated. The expression of AhR in RAW 264.7 macrophages is very low compared to murine AML12 hepatocytes (FIG. 16A). Treatment with AhR inhibitor CH223191 did not alter I3A's effect on reducing palmitate and LPS induced TNFα and IL-β expression in RAW 264.7 macrophages (FIGS. 16B and 16C), indicating that the anti-inflammatory effect of I3A in macrophages does not require AhR activity.

### Example 7: Materials and Methods for Investigating the Effect of I3A to Alleviate Inflammation and Steatosis

Study design. The overall objective of the study was to investigate if I3A can alleviate diet induced liver steatosis and inflammation in vivo. These NAFLD features were induced in mice by feeding the animals a WD and SW for 8 weeks. The drinking water for these mice was then supplemented with low and high doses of I3A, while continuing the WD for another 8 weeks. The phenotypic changes were assessed by measuring body weight gain, serum ALT, hepatic inflammatory cytokines, and histopathological examination of liver tissue. To investigate potential mechanisms of action, a series of omics analyses were performed on fecal material (metagenomics and metabolomics) and liver tissue (metabolomics and proteomics). To determine cell-type specific signaling mediating I3A effects in macrophages, an in vitro cell culture model was used. The histopathological scoring of liver sections was blinded; all other analyses were not blinded. Sample processing and statistical analysis were performed concurrently on treatment and control groups using identical methods. Numbers of replicates and outcomes of statistical tests are indicated in the figure legends.

Materials. RAW 264.7 cells were purchased from ATCC (Manassas, MA). Dulbecco's Modified Eagle Medium (DMEM), penicillin/streptomycin, and LPS (from Salmonella minnesota) were purchased from Invitrogen (Carlsbad, CA). Fetal bovine serum (FBS) was purchased from Atlanta Biologicals (Flowery Branch, GA). All free fatty acid chemicals, 5-aminoimidazole-4-carboxamide ribonucleotide (AICAR) and the AhR inhibitor CH-223191 were purchased from Millipore Sigma (St. Louis, MO). Indole-3-acetate sodium salt (I3A) was purchased from Cayman chemicals (Ann Arbor, MI). All other chemicals were purchased from VWR (Radnor, PA) or Millipore Sigma unless otherwise specified.

Animal experiments. Male B6 129SF1/J mice at 6 weeks of age were obtained from Jackson Laboratories (Bar Harbor, ME). Mice were acclimatized to the animal facility for one week. At the start of the experiment, mice were randomly divided into four groups (n=10 for each group). Three of the four groups were fed ad libitum a Western diet (WD) with 40% Kcal from fat and containing 0.15% cholesterol (D12079B, Research Diets) and a sugar water (SW) solution (23.1 g/L d-fructose+18.9 g/L d-glucose) as previously described (DIAMOND model (15)). After 8 weeks, the three groups of WD-fed mice were randomly selected for treatment with vehicle (WD group) or low (WD-50 group) or high dose (WD-100 group) of I3A. The WD group was fed WD and drank sugar water. The WD-50 and WD-100 groups were fed WD and drank sugar water containing, respectively, 50 or 100 mg I3A per kg body weight. The treatments were continued for another 8 weeks. Water bottles containing SW and I3A or only SW were changed every other day. A fourth group of mice was fed a low-fat control diet (D12450B, Research Diets) and normal water for 16 weeks (CN group) (FIG. 9A). FIG. 9A presents the study design. Mice belonging to the same treatment group were housed together (5 mice/cage). Mice were maintained on 12:12-h light-dark cycles. All procedures were performed in accordance with Texas A&M University Health Sciences Center Institutional Animal Care and Use Committee guidelines under an approved animal use protocol (AUP #2017-0145).

Sample collection. Fecal pellets were collected every other week prior to I3A treatment, right before I3A treatment, every week during I3A treatment, and on the last day of the experiments (FIG. 9B). FIG. 9B presents the sampling scheme. All fecal pellets were flash frozen in liquid nitrogen after collection. Blood samples from the submandibular vein were taken right prior to I3A treatment, every other week during I3A treatment and on the last day of the experiments. The blood samples were centrifuged at 4,000 g for 15 min at 4° C. to obtain serum. At the end of the experiment, the mice were sacrificed by euthanasia. The liver was quickly excised and rinsed with 10× volume of ice-cold PBS. The right medium lobe of the liver was fixed in 10% neutral formalin for histological analysis. A small piece from right lateral lobe was stored in RNAlater (Sigma Aldrich, St. Louis, MO) using the manufacturer's protocol. The remaining liver tissue samples were flash frozen in liquid nitrogen, homogenized in HPLC-grade water, lyophilized to a dry powder, and stored at −80° C. until further processing.

Histological analysis. Formalin fixed liver were embedded in paraffin, sectioned (5 μm) and stained with hematoxylin and eosin (H&E) through VWR histological services (Radnor, PA). Liver histology sections were evaluated by an expert pathologist at Texas A&M University who was blinded to the treatment conditions. Histology was assessed using the NASH CRN and fatty liver inhibition of progression (FLIP) consortia criteria.

Serum ALT and hepatic TG measurement. Alanine aminotransferase (ALT) was measured in serum samples using a commercial ELISA kit (Cayman Chemical Company). Liver triglycerides (TG) were quantified using a commercial colorimetric assay kit (Cayman Chemical Company). Briefly, lyophilized liver samples were weighted and lysed in diluted NP-40 buffer. After centrifugation at 10,000 g and 4° C. for 15 min, the supernatant was stored on ice for quantification of TG. A small amount of lyophilized liver was used for DNA isolation using a DNA miniprep kit (Zymo Research, Irvine, CA). The TG concentrations (mg/dl) were normalized to tissue DNA contents (μg).

Fecal microbiome analysis. Microbial DNA was extracted from homogenized fecal material using the Power soil DNA extraction kit (Qiagen, Carlsbad, CA). The V4 region of 16S rRNA was sequenced on a MiSeq Illumina platform at the Microbial Analysis, Resources, and Services (MARS) core facility (University of Connecticut). Illumina sequence reads were quality filtered, denoised, joined, chimera filtered, aligned, and classified using mothur (v 1.40.4) following the Miseq SOP pipeline. The SILVA database was used for alignment and classification of the operational taxonomic units (OTU) at 97% similarity. The taxonomic dissimilarities between different treatment groups were calculated using the Bray-Curtis dissimilarity metric and visualized on non-metric multidimensional scaling (NMDS) plots. Analysis of similarities (ANOSIM) was used for statistical comparison of microbiome compositions between treatment groups. The differential abundances of OTUs between two different groups were determined using Linear discriminant analysis Effect Size (LEfSe). Fisher's index was calculated to estimate the alpha diversity.

Fecal and serum metabolite extraction. Metabolites were extracted from fecal samples as described previously. Briefly, fecal material was weighed, homogenized, and extracted twice with chloroform/methanol/water. The aqueous phase from the two extractions were combined, lyophilized, and stored at −80° C. Samples were reconstituted in 100 μl methanol/water (1:1, v/v) prior to LC-MS analysis. Serum metabolites were extracted with ice-cold methanol (1:4 serum:methanol). Samples were centrifuged twice at 15,000×g for 5 min at 4° C. The supernatant was stored at −80° C. until LC-MS analysis.

Liver metabolite and protein extraction. Lyophilized liver samples were weighted and homogenized using soft tissue homogenization beads (Omni International) on a bead beater (VWR) with 1 ml ice-cold methanol/water (91:9, v/v). The samples were homogenized for one min, incubated on ice for 5 min, and centrifuged at 12,000×g for 10 min at 4° C. The supernatant was transferred into a new sample tube through a 70 μm cell strainer. Ice-cold chloroform was added into the tube to obtain a final solvent ratio of 47.6/47.6/4.8% methanol/chloroform/water. After vigorous mixing, the samples were frozen in liquid nitrogen and thawed at room temperature. The freeze-thaw cycle was carried out three times. Samples were centrifuged at 15,000×g for 5 min at 4° C. The supernatant and pellet were each transferred into a new sample tube for metabolite and protein analysis, respectively.

For metabolite analysis, 1 ml of HPLC water was added to the supernatant and centrifuged at 10,000×g for 5 min at 4° C. to obtain phase separation. The upper and lower phases were collected separately and filtered through 0.2 μm filters into new sample tubes. The filtered samples were dried to pellets with a lyophilizer and stored at −80° C. until further analysis. The upper and lower phases were reconstituted in 100 μl methanol/water (1:1, v/v) and 200 l methanol, respectively, prior to LC-MS analysis. For protein analysis, the pellet was solubilized in 650 μl extraction buffer (0.5% SDS, 1% v/v β-mercaptoethanol and 1M Tris-HCl, pH=7.6) and 650 μl TRIzol reagent (Thermo Fisher Scientific, Waltham, MA) and incubated at 37° C. for one hour. The sample was centrifuged at 14,000×g for 15 min at 4° C. to obtain phase separation. One ml of ice-cold acetone was mixed into the bottom protein layer. Following an overnight incubation at −20° C., the samples were centrifuged at 14,000×g for 15 min at 4° C. The supernatant was discarded, and the pellet was washed 3× with 1 ml ethanol. The protein pellet was then lyophilized and stored at −80° C. until further analysis.

Untargeted metabolomics. Untargeted LC-MS experiments were performed on a Q Exactive Plus orbitrap mass spectrometer (Thermo Fisher Scientific, Waltham, MA) coupled to a binary pump HPLC system (UltiMate 3000, Thermo Fisher Scientific, Waltham, MA) at the Integrated Metabolomics Analysis Core (IMAC) facility of Texas A&M university as previously described. Chromatographic separation was achieved on a reverse phase (RP) column (Synergi Fusion 4 μm, 150 mm×2 mm, Phenomenex, Torrance, CA) using a gradient method (Table 3).

TABLE 3
Gradient method for untargeted metabolomics

| Time (min) | % Solvent A | % Solvent B |
|---|---|---|
| 0 | 90 | 10 |
| 5 | 60 | 40 |
| 7 | 5 | 95 |
| 9 | 5 | 95 |
| 9.1 | 90 | 10 |
| 13 | 90 | 10 |

Solvent A: 0.1% (v/v) formic acid solution in water. Solvent B was a 0.1% (v/v) formic acid solution in methanol. The flow rate was 0.4 mL/min.

Sample acquisition was performed Xcalibur (Thermo Fisher Scientific, Waltham, MA). Raw data files were processed in Compound Discoverer (version 3.0, Thermo Fisher Scientific, Waltham, MA). Metabolite identification was performed by searching the features against mzCloud and ChemSpider. For comparing the levels of metabolites between samples, the area under the curve (AUC) determined in Compound Discoverer for each feature was normalized to the corresponding sample dry weight.

Liver bile acid analysis. Lyophilized liver samples were homogenized in 10 mM phosphate buffer at pH 6 (28 mg tissue per ml buffer) on a bead beater (VWR, Radnor, PA) for 1 min. Samples were then centrifuged at 15,000×g for 5 min at 4° C. The supernatant (200 μl) was mixed vigorously with 100 ng of d4-glycohenodeoxycholic acid internal standard, 20 μl of saturated ammonium sulfate and 800 μl of acetonitrile, and then centrifuged at 15,000×g for 5 min at 4° C. The supernatant was transferred into a new sample tube and dried with a vacufuge (Eppendorf, Hauppauge, NY). Pellets were resuspended in 100 μl methanol/water (1:1, v/v), vortexed for 30 sec, sonicated for 1 min, and then centrifuged at 15,000 g and 4° C. for 1 min. The supernatant was transferred into a new sample tube and analyzed by LC-MS.

Targeted LC-MS experiments were performed on a TSQ Altis triple quadrupole mass spectrometer (Thermo Fisher Scientific, Waltham, MA) coupled to a binary pump UHPLC (Vanquish, Thermo Scientific) at the TAMU IMAC core facility. Chromatographic separation was achieved on a Kinetex 2.6 μm, 100 mm×2.1 mm polar C18 column (Phenomenex, Torrance, CA) using a gradient method (Table 4). Sample acquisition and data analysis were performed with Trace Finder 4.1 (Thermo Fisher Scientific, Waltham, MA). The calculated bile acid concentrations were normalized to the level of spiked internal standard for each sample.

TABLE 4
Gradient method for bile acid analysis

| Time (min) | % Solvent A | % Solvent B |
|---|---|---|
| 0 | 55 | 45 |
| 9 | 30 | 70 |
| 9.5 | 30 | 70 |
| 9.51 | 55 | 45 |
| 12 | 60 | 40 |

Solvent A: 2 mM ammonium acetate in water. Solvent B: methanol:acetonitrile (50:50, V:V). The flow rate was 0.4 mL/min.

Quantification of free fatty acids. Serum and liver FFAs were analyzed using targeted LC-MS experiments performed on a quadrupole-time of flight instrument (TripleTOF 5600+, AB Sciex, Framingham, MA) as previously described with minor modifications (Tables 2 and 5).

TABLE 5
Gradient method for free fatty acid analysis

| Time (min) | % Solvent A | % Solvent B |
|---|---|---|
| 0 | 90 | 10 |
| 1.7 | 90 | 10 |
| 11.9 | 65 | 35 |
| 14.9 | 0 | 100 |
| 17.4 | 0 | 100 |
| 17.9 | 90 | 10 |
| 20 | 90 | 10 |

Proteins were reduced, alkylated, and digested into peptides using information dependent acquisition (IDA) on a quadrupole-time of flight instrument (TripleTOF 5600+) as previously described with minor modifications (Table 6).

TABLE 6
Gradient method for untargeted proteomics

| Time (min) | % Solvent A | % Solvent B |
|---|---|---|
| 0 | 98 | 2 |
| 15 | 98 | 2 |
| 50 | 55 | 45 |
| 60 | 55 | 45 |
| 62 | 5 | 95 |
| 75 | 5 | 95 |
| 75.5 | 98 | 2 |
| 80 | 98 | 2 |

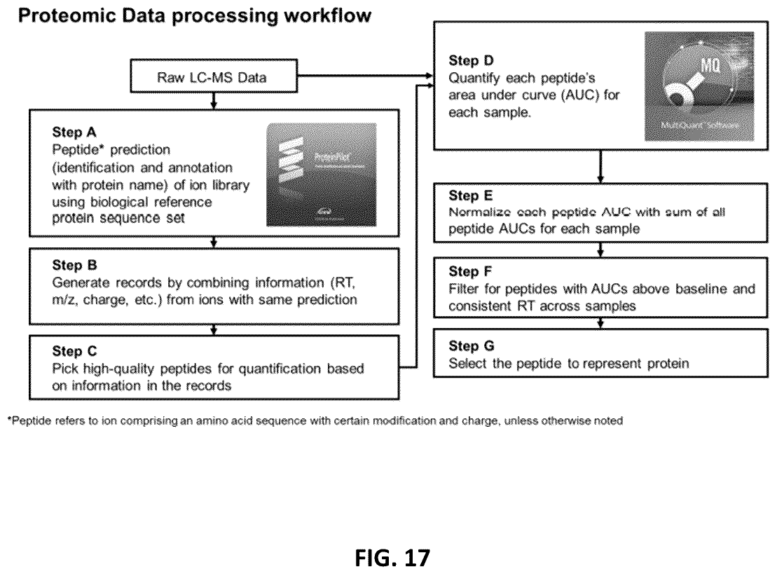

Untargetedproteomics. Peptide ions detected in the IDA experiment were annotated using ProteinPilot (v.5.0, AB Sciex) and further processed using an inhouse script written in MATLAB to associate each protein identified in the sample with a unique high-quality peptide (FIG. 17). The relative abundance of a protein was determined by quantifying the corresponding peptide peak's AUC in Multi-Quant (v2.0, AB) and normalizing the value by the sample's sum of all peptide AUCs.

Targeted proteomics. A panel of 15 proteins having differential abundance in the untargeted proteomics data were selected for targeted measurements using product ion scans for representative peptides (Table 7). The relative abundance of a target protein was determined by quantifying the corresponding peptide peak's AUCs in MultiQuant and normalizing the value by the sample protein weight.

TABLE 7
LC-MS parameters for targeted proteomics

| | | CE | | | RT | Product |
|---|---|---|---|---|---|---|
| Protein | Selected Peptide | (V) | m/z | Charge | (min) | ion(s) |
| CD36 | VAIIESYK | 21.6 | 461.8 | 2 | 32.8 | 752.4 |
| Acox1 | TSNHAIVLAQLITR | 22.6 | 513 | 3 | 37.8 | 701.4 |
| Acaa1a | NTTPDELLSAVLTAVLQDVR | 32.5 | 719.1 | 3 | 53.3 | Sum of |
| | | | | | | 901.5, |
| | | | | | | 1014.6, |
| | | | | | | 1113.7, |
| | | | | | | 800.5, |
| | | | | | | 729.4, |
| | | | | | | 630.4, |
| | | | | | | 517.3 |
| Acaa1b | DTTPDELLSAVLTAVLQDVK | 32.1 | 710.1 | 3 | 54.2 | 873.5 |
| Acads | IGIASQALGIAQASLDCAVK_ | 29.8 | 662.7 | 3 | 42.8 | 792.4 |
| | Carbamidomethyl(C)@17 | | | | | |
| Acadm | LLVEHQGVSFLLAEMAMK | 30.3 | 672.7 | 3 | 42.6 | 793.4 |
| Acad1 | SPAHGISLFL VENGMK | 25.2 | 567.3 | 3 | 39 | 677.3 |
| Hadh | LLVPYLIEAVR | 30.5 | 643.4 | 2 | 43.1 | 960.6 |
| Acaa2 | TNVSGGAIALGHPLGGSGSR | 27 | 603.3 | 3 | 33.5 | 747.4 |
| Fasn | VLEALLPLK | 23.4 | 498.3 | 2 | 39.7 | 783.5 |
| Acab | VEANLLSSEESLSASELSGEQLQEHGDHSCLSYR_ | 45.1 | 941.2 | 4 | 38.1 | 1001.9 |
| | Carbamidomethyl(C)@30 | | | | | |
| Cat | GAGAFGYFEVTHDITR | 25.9 | 580.9 | 3 | 38.1 | 742.7 |
| Gpx1 | YVRPGGGFEPNFTLFEK | 29.4 | 653.3 | 3 | 39.5 | 498.3 |
| Hamp | DTNFPICIFCCK_Carbamidomethyl(C)@7; | 37.6 | 787.8 | 2 | 41.6 | 1097.5 |
| | Carbamidomethyl(C)@10; | | | | | |
| | Carbamidomethyl(C)@11 | | | | | |
| Tf | SAGWVIPIGLLFCK_Carbamidomethyl(C)@13 | 37.3 | 780.9 | 2 | 47 | 947.5 |

CD36: platelet glycoprotein 4; Acox1: peroxisomal acyl-coenzyme A oxidase 1; Acaa1a: peroxisomal 3-ketoacyl-CoA thiolase A; Acaalb: peroxisomal 3-ketoacyl-CoA thiolase B; Acads: short-chain acyl-CoA dehydrogenase; Acadm: medium-chain acyl-CoA dehydrogenase; Acad1: long-chain acyl-CoA dehydrogenase; Hadh: hydroxyacyl-CoA dehydrogenase; Acaa2: mitochondrial 3-ketoacyl-CoA thiolase; Acab: acetyl-CoA carboxylase 2; Cat: catalase; Gpx1: Glutathione peroxidase 1; Hamp: hepcidin; Tf: serotransferrin

Liver cytokine measurements. Lyophilized liver samples were weighted and homogenized with 500 μl lysis buffer (50 mM Tris, 150 mM NaCl, 1% Triton X-100, 1 mM EDTA and 1% protease inhibitor cocktail, PH=7.4) for 1 min on a bead beater (VWR, Radnor, PA). Samples were centrifuged at 20,000×g for 15 min at 4° C. The lipid layer was removed by pipetting and the supernatant was transferred into a new tube. The centrifugation and lipid removal steps were repeated three times. The supernatant was transferred into a new tube and the total protein concentration was measured with the BCA protein assay (Thermo Fisher Scientific, Waltham, MA). A panel of 13 cytokines was quantified with a bead-based ELISA kit (BioLegend, San Diego, CA) following the manufacturer's protocol. Cytokine concentrations (pg/ml) were normalized to the corresponding sample total protein concentration (mg protein/mg tissue).

RAW 264.7 macrophage culture. RAW 264.7 murine macrophages were cultured in a humidified incubator at 37° C. and 5% CO₂ using DMEM supplemented with 10% heat inactivated FBS, penicillin (200 U/mL) and streptomycin (200 μg/mL). Cells were passaged every 2-3 days and used within 10 passages after thawing. For the two-hit model experiment, RAW 264.7 cells were seeded into 24-well plates at a density of 2×105 cells/ml and then treated with 1 mM I3A, followed by palmitate and LPS. For the p-AMPK activation experiment, RAW 264.7 cells were treated with 1 mM AICAR for 4 h, followed by addition of 300 μM palmitate complexed with BSA. Following an 18 h incubation, the cells were treated with 10 ng/ml LPS for another 6 hours.

RNA extraction and qRT-PCR. Total RNA was extracted from RAW 264.7 cell pellets using the EZNA Total RNA kit (Omega Bio-Tek, Norcross, GA). Purity of isolated RNA was confirmed by A260/280 ratio. qRT-PCR analysis was carried out using the qScript One-Step PCR kit (Quanta Biosciences, Gaithersburg, MD) on a LightCycler 96 System (Roche, Indianapolis, IN). Fold-change values were calculated using the 2-ΔΔCt method, with β-actin as the housekeeping gene. The primer sequences are listed in Table 8.

TABLE 8
Primer sequences

| | Forward Primer | Reverse Primer |
|---|---|---|
| Gene | Sequence | Sequence |
| TNFα | TCTCATGCACCACCATCAA | TGACCACTCTCCCTTTGCA |
| | GGACT | GAACT |
| IL-1β | TCCAGGATGAGGACATGAG | GAACGTCACACACCAGCAG |
| | CAC | GTTA |
| β-actin | GGCTGTATTCCCCTCCATCG | CCAGTTGGTAACAATGCCATGT |

AMPK Western blot analysis. Cell pellets or lyophilized liver samples were lysed with modified RIPA buffer (50 mM Tris-HCl, PH 7.4, 1% Triton X-100, 150 mM NaCl, 1 mM EDTA, and 0.5% Sodium deoxycholate) supplemented with a protease inhibitor cocktail (Sigma), 10 mM NaF, and 1 mM Na3VO4. The protein concentration was determined using the BCA protein assay kit (Pierce). Protein samples were denatured with SDS and ˜10 g of protein was separated on a 10% SDS-PAGE gel. Proteins were transferred to a PVDF membrane (Thermo Scientific, Waltham, MA) by wet-transfer electrophoresis. Non-fat milk (5%) in TBST solution was used to block non-specific binding. The blots were probed with appropriate primary antibodies (p-AMPK: 2535, total AMPK: 2603, β-actin: 12620, Cell Signaling Technology) and secondary antibody (Anti-rabbit horseradish peroxidase-conjugated secondary antibody, 7074, Cell Signaling Technology). Proteins bound by both primary and secondary antibodies were visualized by chemiluminescence after incubating the blot with Clarity Max Western ECL Blotting Substrate (Bio-Rad, Hercules, CA). Blot images were acquired on a ChemiDoc gel imaging system (Bio-Rad, Hercules, CA). Proteins were quantified by normalizing the intensity of the protein band of interest to the intensity of the j-actin band in the same lane using the Image Lab software (Bio-Rad, Hercules, CA).

Small interfering RNA transfection. Raw 264.7 cells were seeded into 6-well or 24-well plates at ˜30% confluence and cultured for 24 h prior to transfection. Cells were transfected with ON-TARGETplus mouse prkaa1 siRNA (Dharmacon, Lafayette, CO) or ON-TARGETplus non-targeting pool (negative control, Dharmacon, Lafayette, CO) using the GenMute siRNA transfection reagent (SignaGen Laboratories, Rockville, MD) according to manufacturer's instruction. After 24 h, the medium was replaced with siRNA-free growth medium and incubated for an additional 24 to 72 h. The transfection efficiency was determined by monitoring the AMPK mRNA and protein levels using qRT-PCR and Western blot, respectively.

Statistical analysis. Statistical analysis of the liver metabolomics data was performed using MetaboAnalyst 4.0. Metabolite level comparisons between multiple treatment groups used non-parametric ANOVA. Determination of significant difference between two groups used Wilcoxon rank sum test. Principal component analysis (PCA) and PLS-DA of the metabolomics and proteomics data were conducted using the mixOmics R package (v6.10.9). Ellipses drawn to represent 95% confidence regions assumed Gaussian distribution of latent variables (for PLS-DA) or scores (for PCA). Significance of separation between treatment groups was determined by calculating a standardized Euclidean distance matrix on the coordinates of latent variables or scores and performing a pairwise permutational multivariate analysis of variance (PERMANOVA) with 999 permutations on the distance matrix and Hotelling's T2 tests. A p-value of 0.05 was set as the significance threshold for all statistical comparisons. Heatmaps for liver metabolome (only significantly changed features), fecal metabolome and liver proteome were generated with auto-scaled data. The features in the latter two heatmaps were clustered using the k-means method with Pearson correlation as the similarity metric. For protein analysis, a Variable Importance in Projection (VIP) score was calculated for each protein based on the PLS-DA result, and proteins with a VIP score <1.2 were excluded from the clustering analysis to avoid overfitting. KEGG Pathway enrichment analyses were done using clusterProfiler R package (3.14.3).

Free fatty acid analysis. Free fatty acids (FFAs) in the liver and serum were quantified using product ion scan experiments (TripleTOF 5600+, AB Sciex, Framingham, MA). Chromatographic separation was achieved on a C18 column (Gemini 5 μm C18 110 Å, LC Column 250×2 mm, 5 μm, Phenomenex) using the solvent gradient described in Table 5. Solvent A was acetonitrile/water (3/2, v/v) containing 10 mM ammonium acetate. Solvent B was acetonitrile/isopropanol (1/1, v/v). The injection volume was 5 μL, and the oven temperature was set to 55° C. The flow rate was held at 1 mL/min. The mass spectrometer was operated in negative electrospray ionization (ESI-) mode with MS² scan. The experimental parameters are described in Table 2. “Light” experiments with low collision energy were used for quantification. “Heavy” experiments with high collision energy were used to confirm peak identity. The sample AUCs were converted into absolute concentrations using calibration curves from chemical standards. The FFA concentrations were then normalized by the corresponding sample DNA concentration.

Untargeted proteomics. Proteins extracted from homogenized liver tissue samples were reduced at 37° C. for 30 min using a 50 mM dithiothreitol solution in 50 mM Tris-HCl containing 8 M urea. The reduced proteins were alkylated by incubating the sample in the dark for 15 min with 45.5 mM iodoacetamide. The reduced and alkylated proteins were digested using trypsin. After incubating the sample overnight at 37° C., formic acid was added to lower the pH to 2 and terminate the digest. The sample solution was centrifuged for 5 min at 14,000×g and desalted using spin columns (Pierce, Thermo Fisher). The desalting procedure followed the manufacturer's instruction except that 0.1% (v/v) formic acid was used instead of 0.1% (v/v) trifluoroacetic acid.

Chromatographic separation was achieved on a reverse phase (RP) column (Ascentis Express C18, 2.7 μm 100 Å 15 cm×2.1 mm, Sigma) using a gradient method (Table 6). Solvent A was a 0.1% formic acid solution in water, and solvent B was a 0.1% formic acid solution in acetonitrile. The mobile phase flow rate was held constant at 200 μL/min. The IDA method comprised a TOF MS (survey) scan and (triggered) high-resolution MS/MS (product ion) scans monitoring up to 25 candidate ions per cycle. The dependent scans were triggered whenever the survey scan detected a precursor ion matching the following criteria: mass range of 300-1250 m/z, charge state within +2 to +5, mass tolerance of 50 mDa, ion peak is not an isotope within 6 Da, and ion count exceeds 100 cps. Previously fragmented target ions were excluded for 15 sec to improve the variety of fragmented ions. Collision energy was set to 10 eV.

FIG. 17 is diagram of the untargeted proteomic data analysis workflow. Ions detected in the IDA experiment were annotated using ProteinPilot (v.5.0, AB Sciex) against mouse proteins in the SwissProt database (FIG. 17, Step A). Here, an ion refers to a charged peptide comprising a sequence of amino acids with or without modifications. Ions annotated as the same peptide were collected into one record (Step B). For each record, the average chromatographic retention time (RT), RT variation (relative to average), median peptide confidence score was calculated from the associated ion data. The peptides collected into records were filtered using an automated procedure implemented as a script in MATLAB (R2019b, MathWorks, Natick, MA) based on the following criteria (Step C). For each peptide, average RT and accurate mass in the record were compared with the ions of every other peptide having a different predicted sequence and modification. If a match with another peptide ion was found within 0.1 m/z and 0.5 min, the peptide was excluded from quantification since the overlap decreases confidence in quantitation. All comparisons were performed with ions instead of records because the average RT of a record can be unreliable due to multiple elution of peptides belonging to the same record. The data were analyzed to ensure that a peptide belongs to only one protein, is detected more than once, i.e., at least two ions are included in one record, the median confidence score of a record is greater than 95%, and the maximal difference between RTs of peptide ions within a single record is less than 0.5 min.

Peptides meeting the above criteria were analyzed in MultiQuant (v2.0, AB Sciex) to quantify the areas-under-the curve (AUCs) of corresponding extracted ion chromatograms (XICs) (Step D). The peptide AUCs were then normalized by the corresponding sample's sum of all peptide AUCs to control for sample-to-sample variability in total protein loaded onto the HPLC column (Step E). An additional filter was applied to exclude peptides that did not have AUCs above blank in at least 80% of the samples (Step F). Peptides with RTs that deviate more than 0.5 min among the samples were also excluded from further analysis. To select a representative peptide for protein quantification, peptides having post-translational modification sites were excluded, since the modified peptides can at most represent a fraction of the protein (Step G). If peptides having the same sequence and different charge states were identified for a protein, then the representative peptide was selected from this pool. Otherwise, the representative peptide was selected from all peptides belonging to the protein. In either case, the peptide with the largest average normalized AUC across all samples was selected to represent the corresponding protein's relative abundance.

### Example 8: Depletion of Microbial Trp Metabolites in Severely Ill COVID-19 Patients

A longitudinal study is designed to determine if depletion of the microbial metabolites correlates with or precedes the hyperinflammatory state in severely ill COVID-19 patients. This study design leverages a unique biorepository through which relevant specimens from patients at Tufts Medical Center are collected by trained medical staff from individuals over time, and thus can be used to obtain longitudinal cytokine and metabolite profiles in the context of the individuals' clinical status and treatment record. Combined with rigorous statistical modeling, this allows us to determine if the profile of immunomodulatory metabolite in a patient can forecast hyperinflammation and ARDS. The levels of I3A and TA in patients are evaluated to determine if the I3A and TA profiles correlate with or predict a sustained elevation in inflammatory cytokines in these patients. Through the Tufts COVID-19 Biorepository (https://viceprovost.tufts.edu/tufts-medical-centertufts-university-covid-19-biorepository-and-comprehensive-covid-19-database), various specimens are accessed, including serum, peripheral blood mononuclear cells (PMBCs), and stool samples from inpatients tested for SARS-Cov-2 and treated for COVID-19 at Tufts Medical Center (TMC). These longitudinal specimens from the COVID-19 Biorepository are used to determine the associations between immunomodulatory compounds (I3A and TA), cytokines, and clinical status. These immunomodulatory compounds can be delivered in their original form or as a derivative or as their precursors. To assess if there is a significant depletion in immunomodulatory compounds in ARDS-afflicted COVID-19 patients, the levels of these compounds as present as microbiota metabolites of Trp are evaluated. Defined criteria (respiratory status and proinflammatory markers) are used to assess disease severity. The change in levels of these microbiota metabolites of Trp over the course of illness in a patient are assessed, along with a time-dependent profile of these metabolites in severely ill patients as compared to patients showing moderate symptoms. Statistical (classification and Grainger causality) models are built to determine if a patient's profile of microbiota metabolites can predict one's inflammatory cytokine levels and/or disease severity.

The patient's profile of Trp metabolites is used to forecast the rise or normalization of cytokine levels in the patient, and to determine the correlation with clinical markers of inflammation.

TABLE 9
Summary of Example 8 experimental design

| | |
|---|---|
| Subject groups | Severely ill vs. moderate symptoms (N = 15 per group) |
| Timepoints | Hospitalization day 1, 5, 14, and 21 (are translated into DfSO for |
| | analysis). To the extent possible, subjects are selected who have been |
| | admitted to hospital soon after symptom onset. |
| Disease severity | A patient is classified as 1) severally ill if they are admitted to ICU and |
| | require >6 L/min oxygen support or need mechanical ventilation or 2) |
| | moderately ill if they require less than 3 L/min oxygen support, without |
| | need for ICU admission |
| Clinical markers | C-reactive protein (CRP), ferritin, lactate dehydrogenase (LDH), D-dimer |
| of inflammation | |
| Specimen | Serum, stool, PBMCs will be obtained for each timepoint |
| Assays | Cytokines in serum (multiplex ELISA): virus infection associated |
| | cytokines (IL-1β IL-6, TNF-α) and chemokines (IL-8, MCP-3, CXCL- |
| | 10); inflammasome activation (IL-18); monocyte/T cell activation (IL- |
| | 12, IL-2, LL-7, IL15, IL-17, IL-22, IFNγ) |
| | Trp metabolites in serum and stool (LC-MS): microbial (3A, TA, |
| | indole, indoxyl, indole 3-aldehyde, skatole); host metabolism |
| | (kynurenine, kynurenic acid, xanthurenic acid, cinnabarinic acid) |
| Analysis | Comparison of matched time points for metabolite concentration |
| | (ANOVA) |
| | Comparison of metabolite time profiles (generalized linear mixed |
| | model) |
| | Spearman's rank correlation analysis of metabolite and cytokine time |
| | profiles |
| | Grainger causality test to determine if metabolite profile forecasts |
| | cytokine profile |
| | Random Forest regression to determine which metabolites, if any, are |
| | significant predictors of disease severity |

Subject selection. The subjects (N=15 per group) are selected based on presentation of symptoms and respiratory support status (Table 9). To the greatest extent possible, subjects are selected who have been admitted to hospital soon after symptom onset. Thus, the sampling timepoints are comparable across the two groups, i.e., translate into similar days from symptom onset (DfSO), and the assays will capture the time profiles of metabolites and cytokines before the onset of ARDS.

Clinical status and markers of inflammation. The biorepository provides access to deidentified patient information. These include demographics (age, race, ethnicity, sex), other medical problems (e.g., diabetes), symptoms (respiratory, systemic and GI), days from symptom onset (DfSO), need for ICU admission and mechanical ventilation, laboratory findings (routine chemistry, blood counts, and serum proinflammatory markers), and any treatments received such as steroids, remdesivir, and monoclonal antibodies.

Cytokine assay. A bead-based, multiplexed assay (LEGENDplex, BioLegend) is used to assess correlation of IL-1β, IL-6, TNF-α, IL-8, MCP-3, and CXCL-10 with COVID-19 disease severity, along with the cytokines associated with inflammasome activation (IL-18) and monocyte and T cell activation (IL-12, IL-2, LL-7, IL15, IL-17, IL-22, IFNγ).

Metabolite analysis. The serum and stool samples are extracted at TMC for metabolites using solvent-based methods as described previously. The extraction procedure includes treatments with organic solvents (methanol and chloroform) to ensure inactivation of any virus in the samples. The extracts are analyzed using targeted LC-MS experiments. Trp metabolites shown to bind or activate the AhR are targeted (Table 9). The panel includes products of both host and microbial metabolism. These metabolites are quantified using multiple reaction monitoring (MRM) experiments. High-purity standards are used to optimize the MRM parameters, confirm chromatographic retention time (RT), and generate calibration curves. To account for sample-to-sample and run-to-run variations, respectively, in extraction efficiency and instrument performance, the samples are spiked with isotopically labeled Trp as an internal standard.

Data analysis. A series of statistical analyses is performed to investigate the associations between metabolite profile, cytokine profile, and disease severity. These analyses are applied to both absolute concentrations of metabolites and cytokines as well as data normalized to each subject's day 1 concentrations. For each timepoint, one-way ANOVA is used to test if any of the Trp metabolites are at a significantly different concentration in the severely ill group compared to the moderately ill group. Given that the course of disease varies between individuals within a group, i.e., random effects could contribute to group differences, metabolite trends are compared over time using generalized linear mixed models (GLMMs). A GLMM is built for each Trp metabolite with subject group (severely vs. moderately ill), time, and their interactions as the fixed effects. Bayesian information criterion is used to determine the best-fitting covariance structure. Metabolites showing a significant difference between the two groups based on ANOVA and/or GLMM are further analyzed for correlations with serum cytokines. A Spearman's rank correlation coefficient (rho) is calculated between each pair of cytokine and significant metabolite. Metabolites that correlate significantly (FDR controlled p<0.05) with at least one inflammatory cytokine are included in an autoregression analysis (Grainger causality test) to determine if their time profiles can forecast the time profile of cytokines in serum. These metabolites are included as predictor variables in a Random Forest (RF) regression model on disease severity. This regression is performed on day 1 or 5 metabolite data, as the goal is to develop a predictive model of the hyperinflammatory response that later develops in severely ill COVID-19 patients. The response variable for RF regression is the day 21 clinical status (severely vs. moderately ill) or clinical marker of inflammation (Table 9).

Increased levels of inflammatory cytokines are observed in severely ill subjects, particularly at later timepoints (DfSO 10 or later). A depletion of microbial Trp metabolites in both stool and serum, as both dysregulation of Trp metabolism and gut microbiota dysbiosis has been observed in COVID-19 patients. There may be a lag between depletion of Trp metabolites and elevation in cytokines, which may mask the underlying correlation. On the other hand, the lag may be detectable by the autoregression analysis as discussed here. Different Trp metabolites may be covariates (e.g., due to shared participation in a biochemical reaction or signaling pathway), resulting in redundant variables for the RF regression model. If overfitting of data by the model is detected (e.g., lower validation accuracy), a regularization step (e.g. lasso) is included to select a smaller set of significant variables.

### Example 9: Effect of Immunomodulatory Compounds to Modulate Pro-Inflammatory Cytokine Production in Intestinal Enteroids

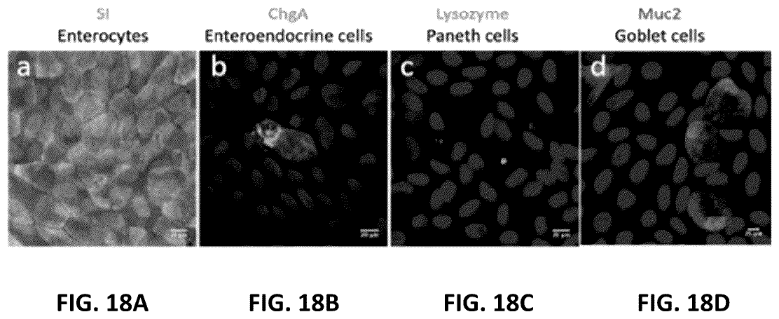

The intestine is a primary site of infection in COVID-19, and viral entry into intestinal epithelial cells occurs through ACE2, mirroring the entry in lung epithelial cells. Human intestinal enteroids (HIEs) express markers of small intestinal epithelial cell differentiation. Deidentified enteroids derived from ileal biopsies of human subjects were obtained from Dr. Mary Estes, Baylor College of Medicine and have been subsequently maintained. Briefly, human intestinal enteroids (HIEs) are thawed from liquid nitrogen storage and cultured at 37° C., 5% CO2 in growth medium containing Wnt 3a, Noggin and R-spondin. HIEs are dissociated into single cells which are seeded onto 0.4 μM transwells and grown as polarized monolayers in growth medium. Monolayers are differentiated by switching to a differentiation medium (growth medium without the addition of Wnt3a, and with 50% reduction in Noggin. Integrity of the monolayers is monitored by transepithelial electrical resistance (TEER) measurements. Differentiated HIE express small intestinal epithelial markers sucrose isomaltase (SI), a marker of enterocytes (FIG. 18A), chromagranin A (ChgA), a marker of enteroendocrine cells (FIG. 18B), lysozyme, a marker of Paneth cells (FIG. 18C), and mucin 2 (Muc2), a marker of goblet cells (FIG. 18D).

Experiments are carried out in cultured human intestinal enteroids (HIEs) to determine if the immunomodulatory compounds (I3A and TA) modulate pro-inflammatory cytokine production in intestinal organoids. These cultured HIEs can be infected with SARS-Cov-2 to evaluate the changes brought on to the production of pro-inflammatory cytokines. The SARS-Cov-2 infected HIE cultures are also used to determine the efficacy of I3A and TA in modulating the infection-induced inflammatory response. This model system is selected because (1) epithelial cells of HIEs express angiotensin-converting enzyme 2 (ACE2), which mediates viral entry into lung cells, and (2) gastrointestinal (GI) symptoms are present in 20% of patients, and in some case are the first and only symptoms. To test if I3A and TA can modulate immune cell-mediated amplification of cytokine signaling, the I3A and TA treatments will be performed in HIEs co-cultured with PMBCs from infected subjects. These experiments will be performed at Tufts New England Regional Biosafety Laboratory (RBL), which has a BSL3 facility supporting SARS-Cov-2 infection studies.

Both I3A and TA dose-dependently downregulate the expression of IL-1, TNF-α, and MCP-1 in certain endotoxin-treated murine bone marrow derived macrophages. As shown here, administration of I3A dissolved in drinking water dose-dependently downregulates hepatic expression of IL-1, TNF-α, MCP-1, IL-6, IL-10, IL-17, and GM-CSF in mice. The epithelial cells of the HIEs express ACE2 and are primary sites of viral infection in COVID-19 patients; thus, supporting the use of cultured HIEs model infected with SARS-Cov-2 as a model. The hyperinflammatory response of severely ill patients develops through recruitment and activation of T cells and monocytes, which can result in widespread inflammation in the lungs and bring about pulmonary edema. To determine if I3A and TA could modulate immune cell mediated amplification of cytokine signaling, the treatments involving I3A and TA are performed in HIEs co-cultured with PBMCs from severely infected subjects. Both I3A and TA are AhR agonists.

Human enteroids (HIEs) are cultured on trans-wells as previously described. For co-culture experiments, previously frozen PBMCs from severely ill patients are thawed and about 5×10⁵ PBMCs per trans-well are added to the basolateral side of HIE containing trans-wells 24 h before introducing viral particles. To mitigate concerns regarding low T cell counts and variability of DfSO between individuals relative to their day of hospitalization, PBMCs isolated from several subjects are pooled to obtain a sufficiently large batch for all co-culture experiments. The PBMC samples are selected based on the subjects' cytokine profile as determined in Example 1. Samples collected at timepoints corresponding to maximal serum concentrations of T helper cell cytokines IL-17A and IL-22 are targeted. A recent longitudinal study found that sustained elevation of these two cytokines is characteristic of severe illness and correlated with increased levels of inflammasome-associated cytokines. SARS-CoV-2 strain stocks are maintained in cold storage at −70° C. in a BSL3 lab. Productive infection and cytopathogenic effect (CPE) are evaluated by microscopy and staining. To achieve infection, SARS-Cov-2 is added to the apical side at a multiplicity of infection (MOI) of 1. A recent study has shown that this MOI results in a productive infection of human small intestinal organoids and inflammatory response within 24 h. The experimental conditions are summarized in Table 10.

A dose response experiment with I3A and TA (20 and 200 μM) is performed. The compounds are added to the apical side of the HIEs in culture medium dissolved in DMSO (<0.1% v/v). Control cultures will receive only the vehicle. The compounds are added to the cultures post-infection (48 h) with fresh medium exchange. Medium samples are collected from the basolateral side of the trans-wells at 24 and 48 h following treatment (72 and 96 h post-infection) and analyzed for cytokines using a multiplexed bead-based assay (Table 10). The dose-response experiments with PBMCs are carried out similarly, except that PBMCs are added to the cultures alongside the metabolites. All experiments are carried out with 5 replicate trans-wells for each condition. Two independent experiments are conducted using different batches of HIEs and PBMCs.

TABLE 10
Experimental Summary

| | | | |
|---|---|---|---|
| | Uninfected | Infected | Infected HIEs + |
| Cultures | HIEs | HIEs | infected PBMCs |

| | |
|---|---|
| Treatments | 13A or TA (20 and 200 μM) or |
| | vehicle control (<0.1% v/v DMSO) |
| | Selective AhR inhibitor (CH-223191) |

| | |
|---|---|
| Samples | Spent medium from basolateral side and HIE |
| | at 24 or 48 h following metabolite treatment |
| | (72 or 96 h post-infection) |
| Assays | Cytokines in spent medium (ELISA); qRT-PCR of |
| | Cyp1b1 and Caspase-1 |
| Analysis | One-way ANOVA for timepoint matched comparisons |
| | between treatment and respective controls |

AhR inhibition and inflammasome activation. The type of immunomodulatory compound and dose combination that results in the greatest attenuation of inflammatory cytokine production are evaluated in HIE and HIE+PBMC cultures. The selective AhR inhibitor CH-223191 will be prepared as a stock solution of 20 mM in DMSO. The infected cultures will be dosed on the apical side with either 1 μL of inhibitor solution or vehicle (0.1% v/v DMSO) 10 min prior to treatment with the immunomodulatory compound. These immunomodulatory compounds are added at different times as described above. Culture medium from the basolateral side and HIEs on trans-wells are collected at 24 and 48 h following metabolite treatment. Inhibition of AhR is confirmed by measuring gene expression using qRT-PCR in cell samples. Inflammasome activation in CH-223191 treated cultures is assessed by measuring NLRP3 expression.

Biochemical assays. Inflammatory cytokines in basolateral medium samples are measured using the same protocol as in Example 1. Activation or inhibition of the AhR by Trp metabolite or CH-223191, respectively, is confirmed by qRT-PCR of Cyp1b1 mRNA. Activation/inhibition of the AhR and inflammasome is assessed by qRT-PCR of Cyp1b1 and Caspase-1 mRNA, respectively.

Data analysis. The Wilcoxon rank-sum test to determine the significance of a treatment compared to its control (e.g., I3A or TA dose vs. vehicle, I3A treatment at 24 vs. 48 h post-infection, I3A plus AhR inhibitor vs. I3A only). Similar to Example 1, time dependence of repeated samples from the same trans-well is analyzed using GLMMs. The in vitro exposure of HIEs to SARS-Cov-2 can result in robust infection and inflammatory response. As an alternative, Caco-2 cells, which are readily infected by SARS-Cov-2, is used. The PBMC co-culture captures tissue-immune cell interactions occurring in vivo. Treatment with the immunomodulatory compounds can attenuate the production of inflammatory cytokines in infected HIEs, including the co-cultures.

The embodiments have been described broadly and generically herein. Each of the narrower species and subgeneric groupings falling within the generic disclosure also form part of the embodiments. It should be understood that although the disclosure contains certain aspects, embodiments, and optional features, modification, improvement and variation of such aspects, embodiments, and optional features can be resorted to by those skilled in the art, and that such modifications, improvements and variations are considered to be within the scope of this disclosure.

What is claimed is:

1. A method of treating a liver disorder, the method comprising administering to a human subject with a liver disorder a therapeutically effective amount of a composition containing a tryptophan metabolite or a derivative thereof.

2. The method of claim 1, wherein the tryptophan metabolite contains indole 3-acetate or a derivative thereof.

3. The method of claim 1, wherein the tryptophan metabolite contains tryptamine or a derivative thereof.

4. The method of claim 1, wherein the tryptophan metabolite contains xanthurenic acid or a derivative thereof.

5. The method of claim 1, wherein the liver disorder is non-alcoholic fatty liver disease, non-alcoholic steatohepatitis, steatosis, and/or fibrosis.

6. The method of claim 2, wherein the liver disorder is non-alcoholic fatty liver disease, non-alcoholic steatohepatitis, steatosis, and/or fibrosis.

7. The method of claim 1, wherein the composition is administered to the human subject orally.

8. The method of claim 1, wherein the composition is formulated as liquid, powder, tablet, paste, capsule, or gel.

9. The method of claim 1, wherein administering the composition to the human subject:
decreases inflammation;
increases liver total bile acids;
decreases primary bile acids; and/or
decreases free fatty acid concentration in liver bile acid.

10. The method of claim 1, wherein administering the composition to the human subject decreases inflammation in the liver, macrophages, the intestine, and/or the lung.

11. A method of treating acute respiratory distress syndrome, the method comprising administering to a human subject a therapeutically effective amount of a composition containing a tryptophan metabolite or a derivative thereof.

12. The method of claim 11, wherein the tryptophan metabolite contains indole 3-acetate or a derivative thereof.

13. The method of claim 11, wherein the tryptophan metabolite contains tryptamine or a derivative thereof.

14. The method of claim 11, wherein the tryptophan metabolite contains xanthurenic acid or a derivative thereof.

15. The method of claim 11, wherein the acute respiratory distress syndrome is associated with an infection by severe acute respiratory syndrome coronavirus 2.

16. The method of claim 12, wherein the acute respiratory distress syndrome is associated with an infection by severe acute respiratory syndrome coronavirus 2.

17. The method of claim 11, wherein the composition is administered to the human subject orally.

18. The method of claim 11, wherein the composition is formulated as liquid, powder, tablet, paste, capsule, or gel.

19. The method of claim 11, wherein administering the composition to the human subject decreases inflammation.

20. The method of claim 11, wherein administering the composition to the human subject decreases inflammation in the liver, macrophages, the intestine, and/or the lung.
